(19) United States
(12) Patent Application Publication
(10) Pub. No.: US 2010/0076021 A1
(43) Pub. Date: Mar. 25, 2010

(54) Organic Compounds

(76) Inventors: Hidetomo Imase, Tsukuba (JP); Masashi Kishida, Tsukuba (JP)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/514,563

(22) PCT Filed: Nov. 13, 2007
(86) PCT No.: PCT/EP2007/062282

§ 371 Date: May 12, 2009

(30) Foreign Application Priority Data
Nov. 15, 2006 (EP) 06124136.0

(51) Int. Cl. A61K 31/4439; C07D 401/12; C07D 401/14; A61K 31/444; A61P 9/00; A61P 3/00

(52) U.S. Cl. 514/318; 546/268.4; 546/194; 514/340

(57) ABSTRACT

The present invention provides a compound of formula (I):

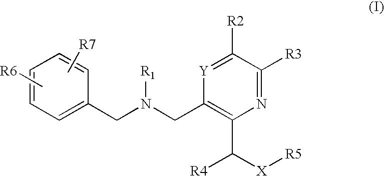

said compound is an inhibitor of CETP, and thus can be employed for the treatment of a disorder or disease mediated by CETP or responsive to the inhibition of CETP.

The present invention related to novel compound of formula (I):

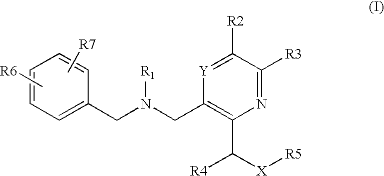

R1 is substituted or unsubstituted heterocyclyl, substituted or unsubstituted aryl, substituted or unsubstituted alkoxycarbonyl, substituted or unsubstituted alkanoyl, or substituted or unsubstituted alkyl;

R2 or R3 are independently of each other hydrogen, substituted or unsubstituted alkyl, substituted or unsubstituted alkoxy, halogen, cyano, nitro, hydroxyl, amino, NR′R″, wherein R′ and R″, independently of one another, represents hydrogen, substituted or unsubstituted alkyl, substituted or unsubstituted aryl, substituted or unsubstituted cycloalkyl, or R′ and R″ form a 5-7-membered carbocyclic ring together with the nitrogen;

or R2 and R3 may form together a 5-7-membered aromatic or heteroaromatic ring fused to the ring to which they are attached, whereby said 5-7-membered aromatic or heteroaromatic ring may be substituted or unsubstituted;

R4 is substituted or unsubstituted alkyl, substituted or unsubstituted aryl, substituted or unsubstituted aryl alkyl or substituted or unsubstituted cycloalkyl;

X is O or NR8;

R5 or R8 are independently of each other hydrogen, substituted or unsubstituted alkyl, substituted or unsubstituted aryl, substituted or unsubstituted cycloalkyl; or

R5 and R8 can form together a 5-7-membered carbocyclic ring together with the nitrogen which ring can be substituted or unsubstituted;

R6 and R7 are independently hydrogen, alkyl, haloalkyl, halogen, cyano, nitro, hydroxy, haloalkoxy, or alkoxy; or

R6 is substituted or unsubstituted aryl or substituted or unsubstituted heteroaryl;

Y is N or CH;

or a pharmaceutically acceptable salt thereof; or an optical isomer thereof; or a mixture of optical isomers.

The present invention also relates to a process for the preparation of these compounds, to the use of these compounds and to pharmaceutical preparations containing such a compound I in free form or in the form of a pharmaceutically acceptable salt.

Extensive pharmacological investigations have shown that the compounds I and their pharmaceutically acceptable salts, for example, have pronounced selectivity in inhibiting CETP (cholesteryl ester transfer protein). CETP is involved in the metabolism of any lipoprotein in living organisms, and has a major role in the reverse cholesterol transfer system. Namely, CETP has drawn attention as a mechanism for preventing accumulation of cholesterol in peripheral cells and preventing arteriosclerosis. In fact, with regard to HDL having an important role in this reverse cholesterol transfer system, a number of epidemiological researches have shown that a decrease in CE (cholesteryl ester) of HDL in blood is one of the risk factors of coronary artery diseases. It has been also clarified that the CETP activity varies depending on the animal species, wherein arteriosclerosis due to cholesterol-loading is hardly induced in animals with lower activity, and in reverse, easily induced in animals with higher activity, and that hyper-HDL-emia and hypo-LDL (low density lipoprotein)-emia are induced in the case of CETP deficiency, thus rendering the development of arteriosclerosis difficult, which in turn led to the recognition of the significance of blood HDL, as well as significance of CETP that mediates transfer of CE in HDL into blood LDL. While many attempts have been made in recent years to develop a drug that inhibits such activity of CETP, a compound having a satisfactory activity has not been developed yet.

For purposes of interpreting this specification, the following definitions will apply and whenever appropriate, terms used in the singular will also include the plural and vice versa.

As used herein, the term “alkyl” refers to a fully saturated branched or unbranched hydrocarbon moiety. Preferably the alkyl comprises 1 to 20 carbon atoms, more preferably 1 to 16 carbon atoms, 1 to 10 carbon atoms, 1 to 7 carbon atoms, or 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl and the like. When an alkyl group includes one or more unsaturated bonds, it can be referred to as an alkenyl (double bond) or an alkynyl (triple bond) group. If the alkyl group can be substituted, it is preferably substituted by 1, 2 or 3 substituents selected from hydroxy, halogen, nitro, carboxy, thiol, cyano, HSO₃—, cycloalkyl, alkenyl, alkoxy, cycloalkoxy, alkenyloxy, alkoxycarbonyl, carbamimidoyl, alkyl-S—, alkyl-SO—, alkyl-SO₂—, amino, H₂N—SO₂—, alkanoyl, or heterocyclyl, more preferably selected from hydroxy, halogen, nitro, carboxy, thiol, cyano, alkoxy, or amino.

The term “aryl” refers to monocyclic or bicyclic aromatic hydrocarbon groups having 6-20 carbon atoms in the ring portion. Preferably, the aryl is a (C₆-C₁₀) aryl. Non-limiting examples include phenyl, biphenyl, naphthyl or tetrahydronaphthyl, most preferably phenyl, each of which may optionally be substituted by 1-4 substituents, such as alkyl, haloalkyl such as trifluoromethyl, cycloalkyl, halogen, hydroxy, alkoxy, alkyl-C(O)—O—, aryl-O—, heteroaryl-O—, amino, acyl, thiol, alkyl-S—, aryl-S—, nitro, cyano, carboxy, alkyl-O—C(O)—, carbamoyl, alkyl-S(O)—, sulfonyl, sulfonamido, heterocyclyl, alkenyl, haloalkoxy, cycloalkoxy, alkenyloxy, alkoxycarbonyl, alkyl-SO—, alkyl-SO₂—, amino, N-mono- or di-substituted (alkyl, cycloalkyl, aryl and/or aryl alkyl) amino or H₂N—SO₂.

Furthermore, the term “aryl” as used herein, refers to an aromatic substituent which can be a single aromatic ring, or multiple aromatic rings that are fused together, linked covalently, or linked to a common group such as a methylene or ethylene moiety. The common linking group also can be a carbonyl as in benzophenone or oxygen as in diphenylether or nitrogen as in diphenylamine.

As used herein, the term “alkoxy” refers to alkyl-O—, wherein alkyl is defined herein above. Representative examples of alkoxy include, but are not limited to, methoxy, ethoxy, propoxy, 2-propoxy, butoxy, tent-butoxy, pentyloxy, hexyloxy, cyclopropyloxy-, cyclohexyloxy- and the like. Preferably, alkoxy groups have about 1-7, more preferably about 1-4 carbons.

As used herein, the term “acyl” refers to a group R—C(O)— of from 1 to 10 carbon atoms of a straight, branched, or cyclic configuration or a combination thereof, attached to the parent structure through carbonyl functionality. Such group can be saturated or unsaturated, and aliphatic or aromatic. Preferably, R in the acyl residue is alkyl, or alkoxy, or aryl, or heteroaryl. When R is alkyl then the moiety is referred to a alkanoyl. Also preferably, one or more carbons in the acyl residue may be replaced by nitrogen, oxygen or sulfur as long as the point of attachment to the parent remains at the carbonyl. Examples include but are not limited to, acetyl, benzoyl, propionyl, isobutyryl, t-butoxycarbonyl, benzyloxycarbonyl and the like. Lower acyl refers to acyl containing one to four carbons.

As used herein, the term “acylamino” refers to acyl-NH—, wherein “acyl” is defined herein.

As used herein, the term “carbamoyl” refers to H₂NC(O)—, alkyl-NHC(O)—, (alkyl)₂NC(O)—, aryl-NHC(O)—, alkyl(aryl)-NC(O)—, heteroaryl-NHC(O)—, alkyl(heteroaryl)-NC(O)—, aryl-alkyl-NHC(O)—, alkyl(aryl-alkyl)-NC(O)— and the like.

As used herein, the term “sulfonyl” refers to R—SO₂—, wherein R is hydrogen, alkyl, aryl, hereoaryl, aryl-alkyl, heteroaryl-alkyl, aryl-O—, heteroaryl-O—, alkoxy, aryloxy, cycloalkyl, or heterocyclyl.

As used herein, the term “sulfonamido” refers to alkyl-S(O)₂—NH—, aryl-S(O)₂—NH—, aryl-alkyl-S(O)₂—NH—, heteroaryl-S(O)₂—NH—, heteroaryl-alkyl-S(O)₂—NH—, alkyl-S(O)₂—N(alkyl)-, aryl-S(O)₂—N(alkyl)-, aryl-alkyl-S(O)₂—N(alkyl)-, heteroaryl-S(O)₂—N(alkyl)-, heteroarrl-alkyl-S(O)₂—N(alkyl)- and the like.

As used herein, the term “alkoxycarbonyl” refers to alkoxy-C(O)—, wherein alkoxy is defined herein.

As used herein, the term “alkanoyl” refers to alkyl-C(O)—, wherein alkyl is defined herein.

As used herein, the term “alkenyl” refers to a straight or branched hydrocarbon group having 2 to 20 carbon atoms and that contains at least one double bonds. The alkenyl groups preferably have about 2 to 8 carbon atoms.

As used herein, the term “alkenyloxy” refers to alkenyl-O—, wherein alkenyl is defined herein.

As used herein, the term “cycloalkoxy” refers to cycloalkoxy-O—, wherein cycloalkyl is defined herein.

As used herein, the term “heterocyclyl” or “heterocycle” refers to an optionally substituted, fully saturated or unsaturated, aromatic or nonaromatic cyclic group, e.g., which is a 4- to 7-membered monocyclic, 7- to 12-membered bicyclic or 10- to 15-membered tricyclic ring system, which has at least one heteroatom in at least one carbon atom-containing ring. Each ring of the heterocyclic group containing a heteroatom may have 1, 2 or 3 heteroatoms selected from nitrogen atoms, oxygen atoms and sulfur atoms, where the nitrogen and sulfur heteroatoms may also optionally be oxidized. The heterocyclic group may be attached at a heteroatom or a carbon atom.

Exemplary monocyclic heterocyclic groups include pyrrolidinyl, pyrrolyl, pyrazolyl, oxetanyl, pyrazolinyl, imidazolyl, imidazolinyl, imidazolidinyl, triazolyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, pyridyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, 1,1,4-trioxo-1,2,5-thiadiazolidin-2-yl and the like.

Exemplary bicyclic heterocyclic groups include indolyl, dihydroidolyl, benzothiazolyl, benzoxazinyl, benzoxazolyl, benzothienyl, benzothiazinyl, quinuclidinyl, quinolinyl, tetrahydroquinolinyl, decahydroquinolinyl, isoquinolinyl, tetrahydroisoquinolinyl, decahydroisoquinolinyl, benzimidazolyl, benzopyranyl, indolizinyl, benzofuryl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]-pyridinyl] or furo[2,3-b]pyridinyl), dihydroisoindolyl, 1,3-dioxo-1,3-dihydroisoindol-2-yl, dihydroquinazolinyl (such as 3,4-dihydro-4-oxo-quinazolinyl), phthalazinyl and the like.

Exemplary tricyclic heterocyclic groups include carbazolyl, dibenzoazepinyl, dithienoazepinyl, benzindolyl, phenanthrolinyl, acridinyl, phenanthridinyl, phenoxazinyl, phenothiazinyl, xanthenyl, carbolinyl and the like.

When heterocyclyl is aromatic, this moiety is referred to as “heteroaryl”.

As used herein, the term “heteroaryl” refers to a 5-14 membered monocyclic- or bicyclic- or fused polycyclic-ring system, having 1 to 8 heteroatoms selected from N, O or S. Preferably, the heteroaryl is a 5-10 membered ring system. Typical heteroaryl groups include 2- or 3-thienyl, 2- or 3-furyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl, 2-, 3-, or 4-pyridyl, 3- or 4-pyridazinyl, 3-, 4-, or 5-pyrazinyl, 2-pyrazinyl, 2-, 4-, or 5-pyrimidinyl.

The term “heteroaryl” also refers to a group in which a heteroaromatic ring is fused to one or more aryl, cycloaliphatic, or heterocyclyl rings, where the radical or point of attachment is on the heteroaromatic ring. Nonlimiting examples include but are not limited to 1-, 2-, 3-, 5-, 6-, 7-, or 8-indolizinyl, 1-, 3-, 4-, 5-, 6-, or 7-isoindolyl, 2-, 3-, 4-, 5-, 6-, or 7-indolyl, 2-, 3-, 4-, 5-, 6-, or 7-indazolyl, 2-, 4-, 5-, 6-, 7-, or 8-purinyl, 1-, 2-, 3-, 4-, 6-, 7-, 8-, or 9-quinolizinyl, 2-, 3-, 4-, 5-, 6-, 7-, or 8-quinoliyl, 1-, 3-, 4-, 5-, 6-, 7-, or 8-isoquinoliyl, 1-, 4-, 5-, 6-, 7-, or 8-phthalazinyl, 2-, 3-, 4-, 5-, or 6-naphthyridinyl, 2-, 3-, 5-, 6-, 7-, or 8-quinazolinyl, 3-, 4-, 5-, 6-, 7-, or 8-cinnolinyl, 2-, 4-, 6-, or 7-pteridinyl, 1-, 2-, 3-, 4-, 5-, 6-, 7-, or 8-4aH carbazolyl, 1-, 2-, 3-, 4-, 5-, 6-, 7-, or 8-carbzaolyl, 1-, 3-, 4-, 5-, 6-, 7-, 8-, or 9-carbolinyl, 1-, 2-, 3-, 4-, 6-, 7-, 8-, 9-, or 10-phenanthridinyl, 1-, 2-, 3-, 4-, 5-, 6-, 7-, 8-, or 9-acridinyl, 1-, 2-, 4-, 5-, 6-, 7-, 8-, or 9-perimidinyl, 2-, 3-, 4-, 5-, 6-, 8-, 9-, or 10-phenathrolinyl, 1-, 2-, 3-, 4-, 6-, 7-, 8-, or 9-phenazinyl, 1-, 2-, 3-, 4-, 6-, 7-, 8-, 9-, or 10-phenothiazinyl, 1-, 2-, 3-, 4-, 6-, 7-, 8-, 9-, or 10-phenoxazinyl, 2-, 3-, 4-, 5-, 6-, or I-, 3-, 4-, 5-, 6-, 7-, 8-, 9-, or 10-benzisoqinolinyl, 2-, 3-, 4-, or thieno[2,3-b]furanyl, 2-, 3-, 5-, 6-, 7-, 8-, 9-, 10-, or 11-7H-pyrazino[2,3-c]carbazolyl, 2-, 3-, 5-, 6-, or 7-2H-furo[3,2-b]-pyranyl, 2-, 3-, 4-, 5-, 7-, or 8-5H-pyrido[2,3-d]-o-oxazinyl, 1-, 3-, or 5-1H-pyrazolo[4,3-d]-oxazolyl, 2-, 4-, or 54H-imidazo[4,5-d]thiazolyl, 3-, 5-, or 8-pyrazino[2,3-d]pyridazinyl, 2-, 3-, 5-, or 6-imidazo[2,1-b]thiazolyl, 1-, 3-, 6-, 7-, 8-, or 9-furo[3,4-c]cinnolinyl, 1-, 2-, 3-, 4-, 5-, 6-, 8-, 9-, 10, or 11-4H-pyrido[2,3-c]carbazolyl, 2-, 3-, 6-, or 7-imidazo[1,2-b][1,2,4]triazinyl, 7-benzo[b]thienyl, 2-, 4-, 5-, 6-, or 7-benzoxazolyl, 2-, 4-, 5-, 6-, or 7-benzimidazolyl, 2-, 4-, 4-, 5-, 6-, or 7-benzothiazolyl, 1-, 2-, 4-, 5-, 6-, 7-, 8-, or 9-benzoxapinyl, 2-, 4-, 5-, 6-, 7-, or 8-benzoxazinyl, 1-, 2-, 3-, 5-, 6-, 7-, 8-, 9-, 10-, or 11-1H-pyrrolo[1,2-b][2]benzazapinyl. Typical fused heteroary groups include, but are not limited to 2-, 3-, 4-, 5-, 6-, 7-, or 8-quinolinyl, 1-, 3-, 4-, 5-, 6-, 7-, or 8-isoquinolinyl, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl.

A heteroaryl group may be mono-, bi-, tri-, or polycyclic, preferably mono-, bi-, or tricyclic, more preferably mono- or bicyclic.

The term “heterocyclyl” further refers to heterocyclic groups as defined herein substituted with 1, 2 or 3 substituents selected from the groups consisting of the following:

alkyl; haloalkyl, hydroxy (or protected hydroxy); halo; oxo, i.e., ═O; amino, N-mono- or di-substituted (alkyl, cycloalkyl, aryl and/or aryl alkyl) amino such as alkylamino or dialkylamino; alkoxy; cycloalkyl; alkenyl; carboxy; heterocyclooxy, wherein heterocyclooxy denotes a heterocyclic group bonded through an oxygen bridge; alkyl-O—C(O)—; mercapto; HSO₃; nitro; cyano; sulfamoyl or sulfonamido; aryl; alkyl-C(O)—O—; aryl-C(O)—O—; aryl-S—; cycloalkoxy; alkenyloxy; alkoxycarbonyl; aryloxy; carbamoyl; alkyl-S—; alkyl-SO—, alkyl-SO₂—; formyl, i.e., HC(O)—; aryl-alkyl-; acyl such as alkanoyl; heterocyclyl and aryl substituted with alkyl, cycloalkyl, alkoxy, hydroxy, amino, alkyl-C(O)—NH—, alkylamino, dialkylamino or halogen.

As used herein, the term “cycloalkyl” refers to optionally substituted saturated or unsaturated monocyclic, bicyclic or tricyclic hydrocarbon groups of 3-12 carbon atoms, each of which may be substituted by one or more substituents, such as alkyl, halo, oxo, hydroxy, alkoxy, alkyl-C(O)—, acylamino, carbamoyl, alkyl-NH—, (alkyl)₂N—, thiol, alkylthio, nitro, cyano, carboxy, alkyl-O—C(O)—, sulfonyl, sulfonamido, sulfamoyl, heterocyclyl and the like. Exemplary monocyclic hydrocarbon groups include, but are not limited to, cyclopropyl, cyclobutyl, cyclopentyl, cyclopentenyl, cyclohexyl and cyclohexenyl and the like. Exemplary bicyclic hydrocarbon groups include bornyl, indyl, hexahydroindyl, tetrahydronaphthyl, decahydronaphthyl, bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, bicyclo[2.2.2]octyl and the like. Exemplary tricyclic hydrocarbon groups include adamantyl and the like.

As used herein, the term “sulfamoyl” refers to H₂NS(O)₂—, alkyl-NHS(O)₂—, (alkyl)₂NS(O)₂—, aryl-NHS(O)₂—, alkyl(aryl)-NS(O)₂—, (aryl)₂NS(O)₂—, heteroaryl-NHS(O)₂—, aralkyl-NHS(O)₂—, heteroaralkyl-NHS(O)₂— and the like.

As used herein, the term “aryloxy” refers to both an —O-aryl and an —O-heteroaryl group, wherein aryl and heteroaryl are defined herein.

As used herein, the term “halogen” or “halo” refers to fluoro, chloro, bromo, and iodo.

As used herein, the term “haloalkyl” refers to an alkyl as defined herein, that is substituted by one or more halo groups as defined herein. Preferably the haloalkyl can be monohaloalkyl, dihaloalkyl or polyhaloalkyl including perhaloalkyl. A monohaloalkyl can have one iodo, bromo, chloro or fluoro within the alkyl group. Dihaloalky and polyhaloalkyl groups can have two or more of the same halo atoms or a combination of different halo groups within the alkyl. Preferably, the polyhaloalkyl contains up to 12, 10, or 8, or 6, or 4, or 3, or 2 halo groups. Non-limiting examples of haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhaloalkyl refers to an alkyl having all hydrogen atoms replaced with halo atoms.

As used herein, the term “isomers” refers to different compounds that have the same molecular formula. Also as used herein, the term “an optical isomer” refers to any of the various stereo isomeric configurations which may exist for a given compound of the present invention and includes geometric isomers. It is understood that a substituent may be attached at a chiral center of a carbon atom. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the term “pharmaceutically acceptable salts” refers to salts that retain the biological effectiveness and properties of the compounds of this invention and, which are not biologically or otherwise undesirable. Non-limiting examples of the salts include non-toxic, inorganic and organic base or acid addition salts of compounds of the present invention. In many cases, the compounds of the present invention are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. The pharmaceutically acceptable salts of the present invention can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts can be found, e.g., in Remington's Pharmaceutical Sciences, 20th ed., Mack Publishing Company, Easton, Pa., (1985), which is herein incorporated by reference.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The term “therapeutically effective amount” of a compound of the present invention refers to an amount of the compound of the present invention that will elicit the biological or medical response of a subject, or ameliorate symptoms, slow or delay disease progression, or prevent a disease, etc. In a preferred embodiment, the “effective amount” refers to the amount that inhibits or reduces expression or activity of CETP.

As used herein, the term “subject” refers to an animal. Preferably, the animal is a mammal. A subject also refers to for example, primates (e.g., humans), cows, sheep, goats, horses, dogs, cats, rabbits, rats, mice, fish, birds and the like. In a preferred embodiment, the subject is a human.

As used herein, the term “a disorder” or “a disease” refers to any derangement or abnormality of function; a morbid physical or mental state. See Dorland's Illustrated Medical Dictionary, (W.B. Saunders Co. 27th ed. 1988).

As used herein, the term “inhibition” or “inhibiting” refers to the reduction or suppression of a given condition, symptom, or disorder, or disease, or a significant decrease in the baseline activity of a biological activity or process. Preferably, the condition or symptom or disorder or disease is mediated by CETP activity or responsive to the inhibition of CETP.

As used herein, the term “treating” or “treatment” of any disease or disorder refers in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

As used herein, the term “a,” “an,” “the” and similar terms used in the context of the present invention (especially in the context of the claims) are to be construed to cover both the singular and plural unless otherwise indicated herein or clearly contradicted by the context. Recitation of ranges of values herein are merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

The following preferred embodiments of the moieties and symbols in formula I can be employed independently of each other to replace more general definitions and thus to define specially preferred embodiments of the invention, where the remaining definitions can be kept broad as defined in embodiments of the inventions defined above of below.

In one embodiment, the invention is related to a compound of formula I wherein

R1 is heterocyclyl, aryl, alkoxycarbonyl, alkanoyl, or alkyl, wherein each heterocyclyl or aryl is optionally substituted with one to three substituents selected from alkyl, haloalkyl, hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, or heterocyclyl; and wherein each alkanoyl, alkoxycarbonyl, or alkyl is optionally substituted with one to three substituents selected from hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, or heterocyclyl;

R2 or R3 are independently of each other hydrogen, alkyl, alkoxy, halogen, cyano, nitro, hydroxyl, amino, NR′R″, wherein R′ and R″, independently of one another, represents hydrogen, alkyl, aryl, cycloalkyl, or R′ and R″ form a 5-7-membered carbocyclic ring together with the nitrogen, wherein each alkyl, alkoxy, aryl or cycloalkyl may be unsubstituted or substituted with one to three substituents selected from haloalkyl, hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, or heterocyclyl,

or R2 and R3 may form together a 5-7-membered aromatic or heteroaromatic ring fused to the ring to which they are attached, whereby said 5-7-membered aromatic or heteroaromatic ring may be unsubstituted or substituted with one to three substituents selected from alkyl, haloalkyl, hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, or heterocyclyl;

R4 is alkyl, aryl, aryl alkyl or cycloalkyl, wherein each alkyl may be unsubstituted or substituted with one to three substituents selected from hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, or heterocyclyl, and wherein each aryl, aryl alkyl or cycloalkyl may be unsubstituted or substituted with one to three substituents selected from alkyl, haloalkyl, hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, or heterocyclyl;

X is O or NR8,

R5 or R8 are independently of each other hydrogen, alkyl, aryl, cycloalkyl, wherein each alkyl may be unsubstituted or substituted with one to three substituents selected from hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, heterocyclyl, or NR′R″, wherein R′ and R″, independently of one another, represents hydrogen, alkyl, aryl or cycloalkyl, or R′ and R″ form a 5-7-membered carbocyclic ring together with the nitrogen, and wherein each aryl, aryl alkyl or cycloalkyl may be unsubstituted or substituted with one to three substituents selected from alkyl, haloalkyl, hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, heterocyclyl, or NR′R″, wherein R′ and R″, independently of one another, represents hydrogen, alkyl, aryl or cycloalkyl, or R′ and R″ form a 5-7-membered carbocyclic ring together with the nitrogen, or

R5 and R8 can form together a 5-7-membered carbocyclic ring together with the nitrogen which ring can be unsubstituted or substituted with one to three substituents selected from alkyl, haloalkyl, hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, heterocyclyl, or NR′R″, wherein R′ and R″ independently of one another, represents hydrogen, alkyl, aryl or cycloalkyl, or R′ and R″ form a 5-7-membered carbocyclic ring together with the nitrogen;

R6 and R7 are independently hydrogen, alkyl, haloalkyl, halogen, cyano, nitro, hydroxy, or alkoxy; or

R6 is aryl or heteroaryl;

Y is CH.

a pharmaceutically acceptable salt thereof; or an optical isomer thereof; or a mixture of optical isomers.

Preferred Definitions for R1

Preferably, R1 is heterocyclyl, aryl, alkoxycarbonyl, alkanoyl, or alkyl, wherein each heterocyclyl or aryl is optionally substituted with one to three substituents selected from alkyl, haloalkyl, hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, or heterocyclyl; and wherein each alkanoyl, alkoxycarbonyl, or alkyl is optionally substituted with one to three substituents selected from hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, or heterocyclyl. More preferably, R1 is s heterocyclyl, alkanoyl or alkoxycarbonyl, wherein each heterocyclyl is optionally substituted with one to three substituents selected from alkyl, hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, or heterocyclyl. It is more preferable that R1 is a 5- or 6-membered, more preferably a 5-membered, N-containing heterocyclce, such as pyrimidyl, pyridyl, pyrazinyl, tetrazoyl, triazoyl, pyrazoyl, or isalkoxycarbonyl, wherein each pyrimidyl, pyridyl, pyrazinyl is optionally substituted with one to three substituents selected from alkyl, hydroxy, halogen, nitro, carboxy, thiol, cyano, HSO₃—, cycloalkyl, alkenyl, alkoxy, cycloalkoxy, alkenyloxy, alkoxycarbonyl, carbamimidoyl, alkyl-S—, alkyl-SO—, alkyl-SO₂—, amino, H₂N—SO₂—, alkanoyl, or heterocyclyl, such as piperidinyl, piperazinyl or morpholinyl.

A preferred meaning of variable R1 is represented by formulae

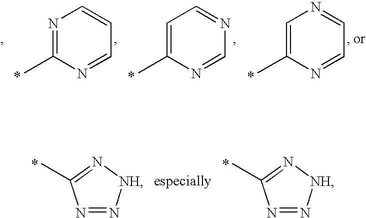

which are each unsubstituted or substituted by C₁-C₄-alkyl, especially methyl or halo, especially methyl.

Preferred Definitions for R2 and R3

Preferably, in one embodiment, R2 or R3 are independently of each other hydrogen, alkyl, alkoxy, halogen, cyano, nitro, hydroxyl, amino, NR′R″, wherein R′ and R″, independently of one another, represents hydrogen, alkyl, aryl, cycloalkyl, or R′ and R″ form a 5-7-membered carbocyclic ring together with the nitrogen, wherein each alkyl, alkoxy, aryl or cycloalkyl may be unsubstituted or substituted with one to three substituents selected from haloalkyl, hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, or heterocyclyl, more preferably, they are independently of each other hydrogen, alkyl, haloalkyl, alkoxy, halogen, cyano, nitro, hydroxyl, amino, NR′R″, wherein R′ and R″, independently of one another, represents hydrogen, alkyl, aryl, cycloalkyl, or R′ and R″ form a 5-7-membered carbocyclic ring together with the nitrogen, preferably R2 or R3 are independently of each other hydrogen or haloalkyl. Preferably, one of R2 and R3, preferably R3, is hydrogen and the other, preferably R2, is a moiety other than hydrogen. Haloalkyl is preferably as defined herein, more preferably fluoromethyl, difluoromethyl or trifluoromethyl, most preferably trifluoromethyl.

In another embodiment, R2 and R3 may form together a 5-7-membered aromatic or heteroaromatic ring fused to the ring to which they are attached, whereby said 5-7-membered aromatic or heteroaromatic ring may be unsubstituted or substituted with one to three substituents selected from alkyl, haloalkyl, hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, or heterocyclyl, preferably they may form together a 5-7-membered aromatic or heteroaromatic ring fused to the ring to which they are attached, whereby said 5-7-membered aromatic or heteroaromatic ring may be unsubstituted or substituted with one to three substituents selected from alkyl, haloalkyl, hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, or heterocyclyl and wherein the aromatic or heteroaromatic ring is selected from phenyl, pyridyl, pyrimidyl, or pyrazinyl. More preferably, the aromatic or heteroaromatic ring is selected from phenyl or pyridyl, most preferably phenyl. If the aromatic or heteroaromatic ring is substituted, it is preferably substituted by alkyl, haloalkyl, hydroxyl or halogen, more preferably halogen such as F.

Preferred Definitions for R4

Preferably R4 is alkyl, aryl, aryl alkyl or cycloalkyl, wherein each alkyl may be unsubstituted or substituted with one to three substituents selected from hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, or heterocyclyl, and wherein each aryl, aryl alkyl or cycloalkyl may be unsubstituted or substituted with one to three substituents selected from alkyl, haloalkyl, hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, or heterocyclyl; preferably, R4 is alkyl or cycloalkyl, wherein alkyl may be unsubstituted or substituted with one to three, preferably one, substituents selected from hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, or heterocyclyl, more preferably hydroxy, halogen, carboxy, alkoxy, cycloalkoxy, alkoxycarbonyl, or carbamoyl, most preferably alkoxycarbonyl.

It is preferred that R4 is methyl, ethyl or n-propyl, more preferably methyl or ethyl. This is particularly the case if X is NR8. It is also preferred that R4 is cyclalkyl such as cyclopentyl or cyclohexyl, preferably cyclohexyl. This is particularly the case if X is O.

Preferred Definitions for X

In one embodiment, X is O.

In another embodiment, X is NR8.

Preferred Definitions for R5 and R8

Preferably, in a first embodiment, R5 or R8 are independently of each other hydrogen, alkyl, aryl, cycloalkyl, wherein each alkyl may be unsubstituted or substituted with one to three substituents selected from hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, heterocyclyl, or NR′R″, wherein R′ and R″, independently of one another, represents hydrogen, alkyl, aryl or cycloalkyl, or R′ and R″ form a 5-7-membered carbocyclic ring together with the nitrogen, and wherein each aryl, aryl alkyl or cycloalkyl may be unsubstituted or substituted with one to three substituents selected from alkyl, haloalkyl, hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, heterocyclyl, or N′R″, wherein R′ and R″, independently of one another, represents hydrogen, alkyl, aryl or cycloalkyl, or R′ and R″ form a 5-7-membered carbocyclic ring together with the nitrogen, more preferably R5 or R8 are independently of each other hydrogen, alkyl or cycloalkyl, wherein each alkyl or cycloalkyl may be unsubstituted or substituted, preferably unsubstituted, with one to three substituents selected from hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, heterocyclyl, or NR′R″, wherein R′ and R″, independently of one another, represents hydrogen, alkyl, aryl or cycloalkyl, or R′ and R″ form a 5-7-membered carbocyclic ring together with the nitrogen; most preferably R5 or R8 are independently of each other hydrogen, methyl, ethyl, cyclopentyl, or cyclohexyl.

It is preferred that ne of R5 and R8 is hydrogen and the other is a moiety other than hydrogen.

In another embodiment, R5 and R8 can form together a 5-7-membered carbocyclic ring together with the nitrogen which ring can be unsubstituted or substituted, preferably unsubstituted, with one to three substituents selected from alkyl, haloalkyl, hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, heterocyclyl, or NR′R″, wherein R′ and R″ independently of one another, represents hydrogen, alkyl, aryl or cycloalkyl, or R′ and R″ form a 5-7-membered carbocyclic ring together with the nitrogen, more preferably alkyl, haloalkyl, hydroxy, halogen, amino, or NR′R″, wherein R′ and R″ independently of one another, represents hydrogen, alkyl, phenyl or cycloalkyl, or R′ and R″ form a pyrrolidinyl or piperidinyl ring together with the nitrogen. More preferably the ring formed by R5 and R8 is selected from a saturated ring, most preferably pyrrolidinyl or piperidinyl.

Preferred Definitions for R6 and R7

Preferably, R6 and R7 are independently hydrogen, alkyl, haloalkyl, halogen, cyano, nitro, hydroxy, or alkoxy; or R6 is aryl or heteroaryl. More preferably, R6 and R7 are independently hydrogen, alkyl, haloalkyl, halogen, or alkoxy. Still more preferably, R6 and R7 are independently hydrogen, alkyl or haloalkyl, such as trifluoromethyl.

In one embodiment, one of R6 and R7 is hydrogen and the other is a group as defined herein other than hydrogen.

In another preferred embodiment, both R6 and R7 are the same and are as defined herein, most preferably trifluoromethyl.

The positions of R6 and R7 on the phenyl ring are preferably as follows:

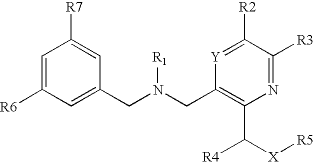

Preferred Definitions for Y

Preferably, Y is CH.

Any asymmetric carbon atom on the compounds of the present invention can be present in the (R)-, (S)- or (R,S)-configuration, preferably in the (R)- or (S)-configuration. Substituents at atoms with unsaturated bonds may, if possible, be present in cis-(Z)- or trans-(E)-form. Therefore, the compounds of the present invention can be in the form of one of the possible isomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, the imidazolyl moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Finally, compounds of the present invention are either obtained in the free form, as a salt thereof, or as prodrug derivatives thereof.

When a basic group is present in the compounds of the present invention, the compounds can be converted into acid addition salts thereof, in particular, acid addition salts with the imidazolyl moiety of the structure, preferably pharmaceutically acceptable salts thereof. These are formed, with inorganic acids or organic acids. Suitable inorganic acids include but are not limited to, hydrochloric acid, sulfuric acid, a phosphoric or hydrohalic acid. Suitable organic acids include but are not limited to, carboxylic acids, such as (C₁-C₄)alkanecarboxylic acids which, for example, are unsubstituted or substituted by halogen, e.g., acetic acid, such as saturated or unsaturated dicarboxylic acids, e.g., oxalic, succinic, maleic or fumaric acid, such as hydroxycarboxylic acids, e.g., glycolic, lactic, malic, tartaric or citric acid, such as amino acids, e.g., aspartic or glutamic acid, organic sulfonic acids, such as (C₁-C₄)alkylsulfonic acids, e.g., methanesulfonic acid; or arylsulfonic acids which are unsubstituted or substituted, e.g., by halogen. Preferred are salts formed with hydrochloric acid, methanesulfonic acid and maleic acid.

When an acidic group is present in the compounds of the present invention, the compounds can be converted into salts with pharmaceutically acceptable bases. Such salts include alkali metal salts, like sodium, lithium and potassium salts; alkaline earth metal salts, like calcium and magnesium salts; ammonium salts with organic bases, e.g., trimethylamine salts, diethylamine salts, tris(hydroxymethyl)methylamine salts, dicyclohexylamine salts and N-methyl-D-glucamine salts; salts with amino acids like arginine, lysine and the like. Salts may be formed using conventional methods, advantageously in the presence of an ethereal or alcoholic solvent, such as a lower alkanol. From the solutions of the latter, the salts may be precipitated with ethers, e.g., diethyl ether. Resulting salts may be converted into the free compounds by treatment with acids. These or other salts can also be used for purification of the compounds obtained.

When both a basic group and an acid group are present in the same molecule, the compounds of the present invention can also form internal salts.

The present invention also provides pro-drugs of the compounds of the present invention that converts in vivo to the compounds of the present invention. A pro-drug is an active or inactive compound that is modified chemically through in vivo physiological action, such as hydrolysis, metabolism and the like, into a compound of this invention following administration of the prodrug to a subject. The suitability and techniques involved in making and using pro-drugs are well known by those skilled in the art. Prodrugs can be conceptually divided into two non-exclusive categories, bioprecursor prodrugs and carrier prodrugs. See The Practice of Medicinal Chemistry, Ch. 31-32 (Ed. Wermuth, Academic Press, San Diego, Calif., 2001). Generally, bioprecursor prodrugs are compounds are inactive or have low activity compared to the corresponding active drug compound, that contains one or more protective groups and are converted to an active form by metabolism or solvolysis. Both the active drug form and any released metabolic products should have acceptably low toxicity. Typically, the formation of active drug compound involves a metabolic process or reaction that is one of the follow types:
1. Oxidative reactions, such as oxidation of alcohol, carbonyl, and acid functions, hydroxylation of aliphatic carbons, hydroxylation of alicyclic carbon atoms, oxidation of aromatic carbon atoms, oxidation of carbon-carbon double bonds, oxidation of nitrogen-containing functional groups, oxidation of silicon, phosphorus, arsenic, and sulfur, oxidative N-delakylation, oxidative O— and S-delakylation, oxidative deamination, as well as other oxidative reactions.
2. Reductive reactions, such as reduction of carbonyl groups, reduction of alcoholic groups and carbon-carbon double bonds, reduction of nitrogen-containing functions groups, and other reduction reactions.
3. Reactions without change in the state of oxidation, such as hydrolysis of esters and ethers, hydrolytic cleavage of carbon-nitrogen single bonds, hydrolytic cleavage of non-aromatic heterocycles, hydration and dehydration at multiple bonds, new atomic linkages resulting from dehydration reactions, hydrolytic dehalogenation, removal of hydrogen halide molecule, and other such reactions.
Carrier prodrugs are drug compounds that contain a transport moiety, e.g., that improve uptake and/or localized delivery to a site(s) of action. Desirably for such a carrier prodrug, the linkage between the drug moiety and the transport moiety is a covalent bond, the prodrug is inactive or less active than the drug compound, and any released transport moiety is acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. See, Cheng et al., US20040077595, application Ser. No. 10/656,838, incorporated herein by reference. Such carrier prodrugs are often advantageous for orally administered drugs. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of hydroxy groups with lipophilic carboxylic acids, or of carboxylic acid groups with alcohols, e.g., aliphatic alcohols. Wermuth, The Practice of Medicinal Chemistry, Ch. 31-32, Ed. Werriuth, Academic Press, San Diego, Calif., 2001.

Exemplary prodrugs are, e.g., esters of free carboxylic acids and S-acyl and O-acyl derivatives of thiols, alcohols or phenols, wherein acyl has a meaning as defined herein. Preferred are pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the ω-(amino, mono- or di-lower alkylamino, carboxy, lower alkoxycarbonyl)-lower alkyl esters, the α-(lower alkanoyloxy, lower alkoxycarbonyl or di-lower alkylaminocarbonyl)-lower alkyl esters, such as the pivaloyloxymethyl ester and the like conventionally used in the art. In addition, amines have been masked as arylcarbonyloxymethyl substituted derivatives which are cleaved by esterases in vivo releasing the free drug and formaldehyde (Bundgaard, J. Med. Chem. 2503 (1989)). Moreover, drugs containing an acidic NH group, such as imidazole, imide, indole and the like, have been masked with N-acyloxymethyl groups (Bundgaard, Design of Prodrugs, Elsevier (1985)). Hydroxy groups have been masked as esters and ethers. EP 039,051 (Sloan and Little) discloses Mannich-base hydroxamic acid prodrugs, their preparation and use.

In view of the close relationship between the compounds, the compounds in the form of their salts and the pro-drugs, any reference to the compounds of the present invention is to be understood as referring also to the corresponding pro-drugs of the compounds of the present invention, as appropriate and expedient.

Furthermore, the compounds of the present invention, including their salts, can also be obtained in the form of their hydrates, or include other solvents used for their crystallization. The compounds of the present invention have valuable pharmacological properties. The compounds of the present invention are useful as inhibitors for cholesteryl ester transfer protein (CETP). CETP is a 74 KD glycopeptide, it is secreted by the liver and is a key player in facilitating the transfer of lipids between the various lipoproteins in plasma. The primary function of CETP is to redistribute cholesteryl esters (CE) and triglycerides between lipoproteins. See Assmann, G et al., “HDL cholesterol and protective factors in atherosclerosis,” Circulation, 109: 1118-1114 (2004). Because most triglycerides in plasma originate in VLDLs and most CEs are formed in HDL particles in the reaction catalyzed by lecithin:cholesterol acyltransferase, activity of CETP results in a net mass transfer of triglycerides from VLDLs to LDLs and HDLs and a net mass transfer of CEs from HDLs to VLDLs and LDLs. Thus, CETP potentially decreases HDL-C levels, increases LDL-cholesteryl (LDL-C) levels and reduces HDL and LDL particles size, and inhibition of CETP could be a therapeutic strategy for raising HDL-cholesteryl (HDL-C), have a favorable impact on the lipoprotein profile, and reduce the risk of cardiovascular diseases. Accordingly, the compounds of the present invention as CETP inhibitors are useful for the delay of progression and/or treatment of a disorder or disease that is mediated by CETP or responsive to inhibition of CETP. Disorders, conditions and diseases that can be treated with the compounds of the present invention include but are not limited to, hyperlipidemia, arteriosclerosis, atherosclerosis, peripheral vascular disease, dyslipidemia, hyperbetalipoproteinemia, hypoalphalipoproteinemia, hypercholesterolemia, hypertriglyceridemia, familial hypercholesterolemia, cardiovascular disorder, coronary heart disease, coronary artery disease, coronary vascular disease, angina, ischemia, heart ischemia, thrombosis, cardiac infarction such as myocardial infarction, stroke, peripheral vascular disease, reperfusion injury, angioplasty restenosis, hypertension, congestive heart failure, diabetes such as type II diabetes mellitus, diabetic vascular complications, obesity, infection or egg embryonation of schistosoma, or endotoxemia etc.

Additionally, the present invention provides:
a compound of the present invention as described herein above for use as a medicament;
the use of a compound of the present invention as described herein above for the preparation of a pharmaceutical composition for the delay of progression and/or treatment of a disorder or disease mediated by CETP, or responsive to inhibition of CETP.
the use of a compound of the present invention as described herein above for the preparation of a pharmaceutical composition for the delay of progression and/or treatment of a disorder or disease selected from hyperlipidemia, arteriosclerosis, atherosclerosis, peripheral vascular disease, dyslipidemia, hyperbetalipoproteinemia, hypoalphalipoproteinemia, hypercholesterolemia, hypertriglyceridemia, familial hypercholesterolemia, cardiovascular disorder, coronary heart disease, coronary artery disease, coronary vascular disease, angina, ischemia, heart ischemia, thrombosis, cardiac infarction such as myocardial infarction, stroke, peripheral vascular disease, reperfusion injury, angioplasty restenosis, hypertension, congestive heart failure, diabetes such as type II diabetes mellitus, diabetic vascular complications, obesity or endotoxemia etc.

The compounds of formula (I) can be prepared by the procedures described in the following sections.

Generally, the compounds of formula (I) can be prepared according to the following general procedures and schemes. In all these Schemes the variants R1, R2, R3, R4, R5, R6, R7 and R8, X and Y have the meaning as set forth herein unless defined otherwise.

General Synthesis of Compounds of Formula (I) is Outlined in the Following Schemes:

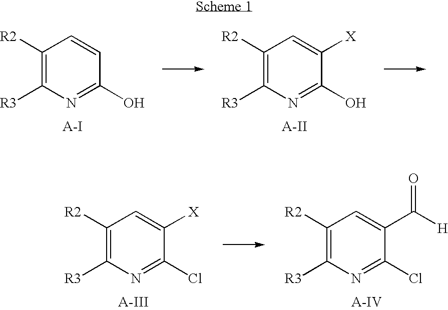

Starting from pyridone (A-I), halogenation with an appropriate reagent such as N-bromosuccinimide and bromine at −20˜30° C. in inert solvents such as dichloromethane gives compound A-II. Treatment with an appropriate reagent such as phosphoryl chloride at −20˜30° C. affords compound A-III. Halogen-metal exchange can be performed with alkyl metal reagents such as n-butyl lithium, and formaylation with a formylating agent such as N,N-dimethylformamide gives compound A-IV.

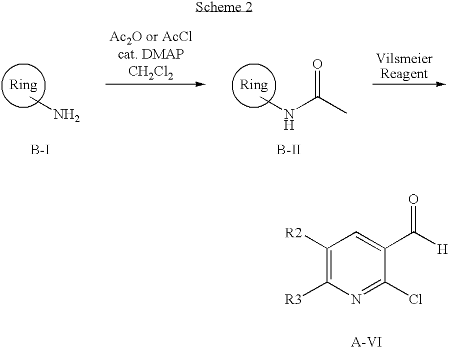

Compound A-VI can be prepared from compound B-I or B-II, which can be purchased or prepared as shown in Scheme 1. An appropriately substituted aryl amine B-I is treated with acetic anhydride (Ac₂O) or acetyl chloride (AcCl) with catalytic amount of 4-N,N-dimethylaminopyridine (DMAP) in CH₂Cl₂ to afford the corresponding compound B-II. Vilsmeier-type cyclization of compound B-II by treatment with phosphoryl chloride (POCl₃) in DMF gave the corresponding compound A-VI [see for example: Meth-Cohn et al., J. Chem. Soc., Perkin Trans. 1 1520 (1981)].

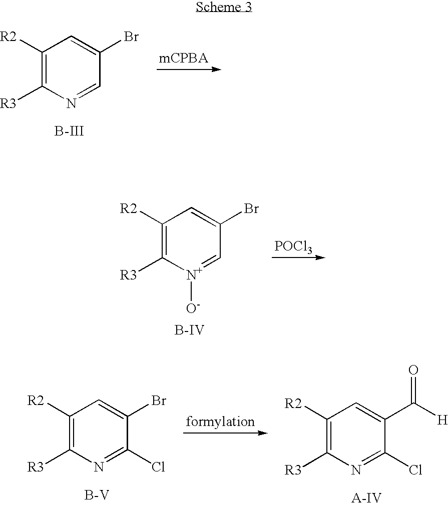

Alternatively, compound A-IV can be prepared from compound B-III. An appropriately substituted aryl bromide B-III is treated with m-chloroperbenzoic acid (m-CPBA) in CH₂Cl₂ to afford the corresponding intermediates B-IV. Chlorination of intermediates B-IV by treatment with phosphoryl chloride (POCl₃) may give the corresponding intermediates B-V [see for example: Grig-Alexa et al., Synlett 11, 2000 (2004)]. Conversion of bromine atom in intermediates B-V to formyl group may be accomplished with n-BuLi and DMF to give compound A-IV. Alternatively, formylation can be employed with carbon monoxide and sodium formate or hydrogen, in the presence of palladium catalyst [see for example: Okano et al., Bull. Chem. Soc. Jap. 67, 2329 (1994)].

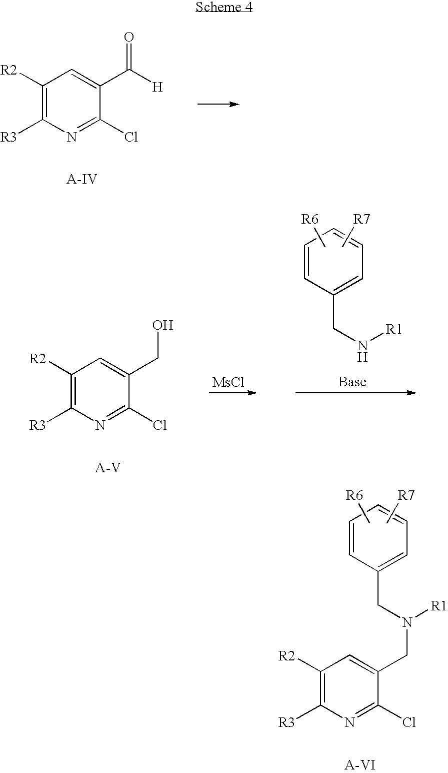

Reduction of aldehyde group by using a reducing reagent such as sodium borohydride or lithium aluminum hydride gives the corresponding alcohol (A-V). After conversion of alcohol group to a leaving group, for example, conversion to methanesulfonate, chloride or bromide, a secondary amine can be alkylated in the presence of a base such as diisopropylethylamine, triethylamine or potassium carbonate to give Compound A-VII.

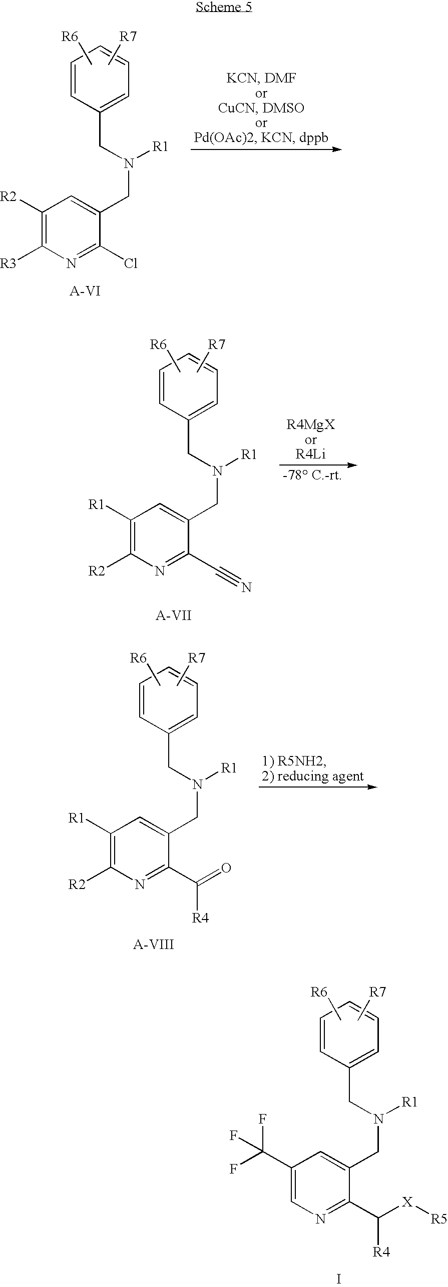

The desired compound A-VII may be prepared from compound A-VI by treatment with nucleophile agents, such as potassium cyanide or cupper cyanide in a solvent such as dimethylformamide or dimethylsulfoxide at 100-180° C., typically 110° C. At times, the reaction is carried out by using palladium acetate as a catalyst. The product is usually isolated by standard extractive work up and flash chromatography on silica gel. The desired compound A-VIII may be prepared from the corresponding compound A-VII by reaction with appropriate reagents, such as alkyl lithium or Grignard reagent in a solvent such as tetrahydrofuran or diethylether at −78° C.-rt, typically −78° C. After acidic workup, the product is usually isolated by standard extractive work up and flash chromatography on silica gel. A8-A7 (2-3)

The desired compound I may be prepared from compound A-VIII by a reductive amination sequence. The compound A-VIII and an excess of amine (preferably 1.1 equivalents) in a polar solvent (preferably dichloromethane) are treated with acidic reagent, such as titanium tetrachloride, acetic acid or boron trifluoride at temperature between about 0° C. to 40° C. The resulting imine is reduced by treatment with a reducing reagent (preferably sodium triacetoxy borohydride) in an appropriate polar solvent (preferably ethanol) at a temperature between 0° C. to 80° C. (preferably room temperature) to provide the desired compound I.

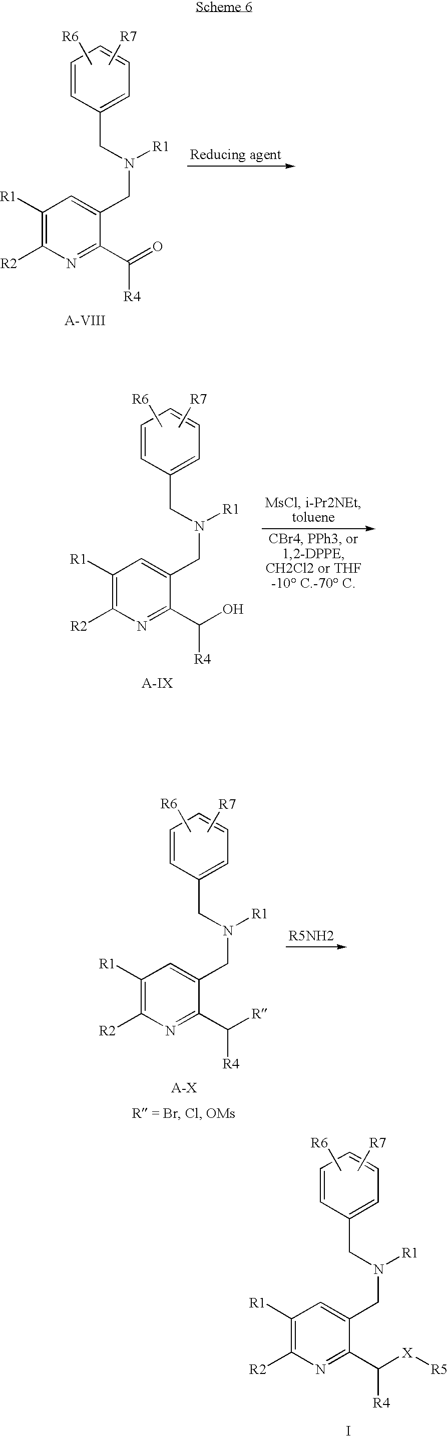

The desired compound A-IX may be prepared from the corresponding compound A-VIII by treatment with a reducing reagent, such as lithium aluminum hydride, sodium borohydride or sodium triacetoxy borohydride (preferably sodium borohydride) in a solvent such as tetrahydrofuran or alcohol (preferably ethanol) at a temperature between 0° C. to 80° C. (preferably room temperature) to provide the desired compound A-IX.

The desired compound A-X may be prepared from the corresponding compound A-IX by treatment with a appropriate reagent, such as p-toluenesulfonyl chloride or methanesufonyl chloride (preferably methanesulfonyl chloride) and an excess (preferably 3 equivalents) of a base (preferably diisopropylethylamine) in a solvent (preferably toluene) at a temperature between 0° C. to 40° C. (preferably room temperature) to provide the desired compound. Alternatively, the desired compound A-X may be prepared from the corresponding compound A-IX by reaction with halogenating reagents (preferably carbon tetrabromide) and phosphine reagent, such as triphenylphosphine or 1,2-diphenylphosphinoethane (preferably triphenylphosphine) in a solvent (preferably tetrahydrofuran) at a temperature between −10° C. to 70° C. (preferably room temperature) to provide the desired compound.

The desired compound I may be prepared from the corresponding compound A-X by reaction with appropriate amine (R5NH2) in a solvent (preferably dimethylsulfoxide) at a temperature between 0° C. to 80° C. (preferably room temperature) to provide the desired compound.

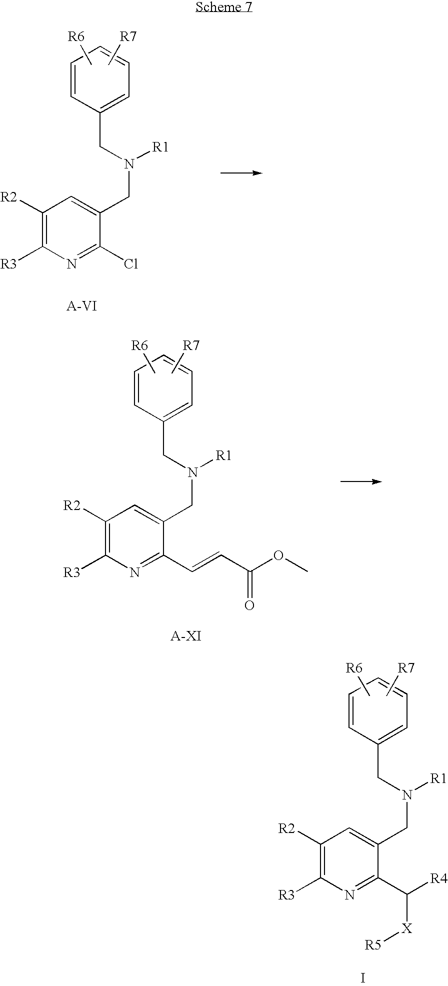

The desired compound A-XI may be prepared from the corresponding compound A-VII by Heck reaction sequence. The compound A-VI and an excess of methyl acrylate (preferably 2 equivalents) in a polar solvent (preferably dimethylformamide) are treated with catalytic amount (preferably 0.1 equivalent) of palladium, such as palladium acetate at temperature between about 80° C. to 140° C. (preferably 120° C.) to provide the desired compound.

The compound A-XI and an excess (preferably 10 equivalents) of an appropriate amine in a solvent (preferably tetrahydrofuran) are treated with an excess (preferably 2 equivalent) of Lewis acid (preferably lithium perchlorite) at temperature between about 0° C. to room temperature (preferably ambient temperature) to provide the desired compound I.

Racemates and diastereomer mixtures obtained can be separated into the pure isomers or racemates in a known manner on the basis of the physicochemical differences of the components, for example by fractional crystallization or by chiral chromotagraphy or HPLC separation utilizing chiral stationery phases. Racemates obtained may furthermore be resolved into the optical antipodes by known methods, for example by recrystallization from an optically active solvent, chromatography on chiral adsorbents, with the aid of suitable microorganisms, by cleavage with specific immobilized enzymes, via the formation of inclusion compounds, for example using chiral crown ethers, only one enantiomer being complexed, or by conversion into diastereomeric salts, for example by reaction of a basic final substance racemate with an optically active acid, such as a carboxylic acid, for example tartaric or malic acid, or sulfonic acid, for example camphorsulfonic acid, and separation of the diastereomer mixture obtained in this manner, for example on the basis of its differing solubilities, into the diastereomers from which the desired enantiomer can be liberated by the action of suitable agents. The more active enantiomer is advantageously isolated.

In starting compounds and intermediates which are converted to the compounds of the invention in a manner described herein, functional groups present, such as amino, thiol, carboxyl and hydroxy groups, are optionally protected by conventional protecting groups that are common in preparative organic chemistry. Protected amino, thiol, carboxyl and hydroxy groups are those that can be converted under mild conditions into free amino thiol, carboxyl and hydroxy groups without the molecular framework being destroyed or other undesired side reactions taking place.

The purpose of introducing protecting groups is to protect the functional groups from undesired reactions with reaction components under the conditions used for carrying out a desired chemical transformation. The need and choice of protecting groups for a particular reaction is known to those skilled in the art and depends on the nature of the functional group to be protected (hydroxy group, amino group, etc.), the structure and stability of the molecule of which the substituent is a part and the reaction conditions.

Well-known protecting groups that meet these conditions and their introduction and removal are described, e.g., in McOmie, “Protective Groups in Organic Chemistry”, Plenum Press, London, NY (1973); and Greene and Wuts, “Protective Groups in Organic Synthesis”, John Wiley and Sons, Inc., NY (1999).

The above-mentioned reactions are carried out according to standard methods, in the presence or absence of diluent, preferably, such as are inert to the reagents and are solvents thereof, of catalysts, condensing or said other agents, respectively and/or inert atmospheres, at low temperatures, room temperature or elevated temperatures, preferably at or near the boiling point of the solvents used, and at atmospheric or super-atmospheric pressure. The preferred solvents, catalysts and reaction conditions are set forth in the appended illustrative Examples.

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure antipodes.

Compounds of the invention and intermediates can also be converted into each other according to methods generally known per se.

In another aspect, the present invention provides a pharmaceutical composition comprising a compound of the present invention and a pharmaceutically acceptable carrier. The pharmaceutical composition can be formulated for particular routes of administration such as oral administration, parenteral administration, and rectal administration, etc. In addition, the pharmaceutical compositions of the present invention can be made up in a solid form including capsules, tablets, pills, granules, powders or suppositories, or in a liquid form including solutions, suspensions or emulsions. The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifers and buffers etc.

Preferably, the pharmaceutical compositions are tablets and gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Injectable compositions are preferably aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, preferably about 1-50%, of the active ingredient.

Suitable compositions for transdermal application include an effective amount of a compound of the invention with carrier. Advantageous carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

The present invention further provides anhydrous pharmaceutical compositions and dosage forms comprising the compounds of the present invention as active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e. g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The invention likewise relates to a combination of a compound of formula (I), (I A) or (I B), respectively, or a pharmaceutically acceptable salt thereof with a further active principle.

The combination may be made for example with the following active principles, selected from the group consisting of a:
(i) HMG-Co-A reductase inhibitor or a pharmaceutically acceptable salt thereof,
(ii) angiotensin II receptor antagonist or a pharmaceutically acceptable salt thereof,
(iii) angiotensin converting enzyme (ACE) Inhibitor or a pharmaceutically acceptable salt thereof,
(iv) calcium channel blocker or a pharmaceutically acceptable salt thereof,
(v) aldosterone synthase inhibitor or a pharmaceutically acceptable salt thereof,
(vi) aldosterone antagonist or a pharmaceutically acceptable salt thereof,
(vii) dual angiotensin converting enzyme/neutral endopeptidase (ACE/NEP) inhibitor or a pharmaceutically acceptable salt thereof,
(viii) endothelin antagonist or a pharmaceutically acceptable salt thereof,
(ix) renin inhibitor or a pharmaceutically acceptable salt thereof,
(x) diuretic or a pharmaceutically acceptable salt thereof, and
(xi) an ApoA-I mimic.

An angiotensin II receptor antagonist or a pharmaceutically acceptable salt thereof is understood to be an active ingredients which bind to the AT₁-receptor subtype of angiotensin II receptor but do not result in activation of the receptor. As a consequence of the inhibition of the AT₁ receptor, these antagonists can, for example, be employed as antihypertensives or for treating congestive heart failure.

The class of AT₁ receptor antagonists comprises compounds having differing structural features, essentially preferred are the non-peptidic ones. For example, mention may be made of the compounds which are selected from the group consisting of valsartan, losartan, candesartan, eprosartan, irbesartan, saprisartan, tasosartan, telmisartan, the compound with the designation E-1477 of the following formula

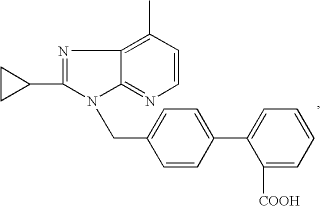

the compound with the designation SC-52458 of the following formula

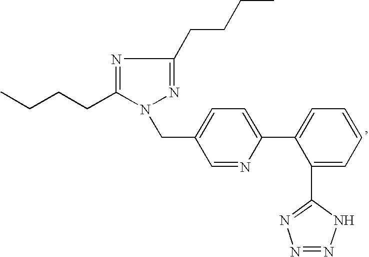

and the compound with the designation ZD-8731 of the following formula

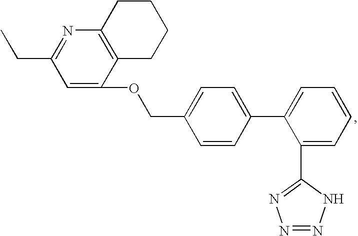

or, in each case, a pharmaceutically acceptable salt thereof.

Preferred AT₁-receptor antagonist are those agents which have been marketed, most preferred is valsartan or a pharmaceutically acceptable salt thereof.

HMG-Co-A reductase inhibitors (also called □-hydroxy-□-methylglutaryl-co-enzyme-A reductase inhibitors) are understood to be those active agents that may be used to lower the lipid levels including cholesterol in blood.

The class of HMG-Co-A reductase inhibitors comprises compounds having differing structural features. For example, mention may be made of the compounds that are selected from the group consisting of atorvastatin, cerivastatin, compactin, dalvastatin, dihydrocompactin, fluindostatin, fluvastatin, lovastatin, pitavastatin, mevastatin, pravastatin, rivastatin, simvastatin, and velostatin, or, in each case, a pharmaceutically acceptable salt thereof.

Preferred HMG-Co-A reductase inhibitors are those agents which have been marketed, most preferred is fluvastatin and pitavastatin or, in each case, a pharmaceutically acceptable salt thereof.

The interruption of the enzymatic degradation of angiotensin I to angiotensin II with so-called ACE-inhibitors (also called angiotensin converting enzyme inhibitors) is a successful variant for the regulation of blood pressure and thus also makes available a therapeutic method for the treatment of congestive heart failure.

The class of ACE inhibitors comprises compounds having differing structural features. For example, mention may be made of the compounds which are selected from the group consisting alacepril, benazepril, benazeprilat, captopril, ceronapril, cilazapril, delapril, enalapril, enaprilat, fosinopril, imidapril, lisinopril, moveltopril, perindopril, quinapril, ramipril, spirapril, temocapril, and trandolapril, or, in each case, a pharmaceutically acceptable salt thereof.

Preferred ACE inhibitors are those agents that have been marketed, most preferred are benazepril and enalapril.

The class of CCBs essentially comprises dihydropyridines (DHPs) and non-DHPs such as diltiazem-type and verapamil-type CCBs.

A CCB useful in said combination is preferably a DHP representative selected from the group consisting of amlodipine, felodipine, ryosidine, isradipine, lacidipine, nicardipine, nifedipine, niguldipine, niludipine, nimodipine, nisoldipine, nitrendipine, and nivaldipine, and is preferably a non-DHP representative selected from the group consisting of flunarizine, prenylamine, diltiazem, fendiline, gallopamil, mibefradil, anipamil, tiapamil and verapamil, and in each case, a pharmaceutically acceptable salt thereof. All these CCBs are therapeutically used, e.g. as anti-hypertensive, anti-angina pectoris or anti-arrhythmic drugs.

Preferred CCBs comprise amlodipine, diltiazem, isradipine, nicardipine, nifedipine, nimodipine, nisoldipine, nitrendipine, and verapamil, or, e.g. dependent on the specific CCB, a pharmaceutically acceptable salt thereof. Especially preferred as DHP is amlodipine or a pharmaceutically acceptable salt, especially the besylate, thereof. An especially preferred representative of non-DHPs is verapamil or a pharmaceutically acceptable salt, especially the hydrochloride, thereof.

Aldosterone synthase inhibitor is an enzyme that converts corticosterone to aldosterone to by hydroxylating cortocosterone to form 18-OH-corticosterone and 18-OH-corticosterone to aldosterone. The class of aldosterone synthase inhibitors is known to be applied for the treatment of hypertension and primary aldosteronism comprises both steroidal and non-steroidal aldosterone synthase inhibitors, the later being most preferred.

Preference is given to commercially available aldosterone synthase inhibitors or those aldosterone synthase inhibitors that have been approved by the health authorities.

The class of aldosterone synthase inhibitors comprises compounds having differing structural features. For example, mention may be made of the compounds which are selected from the group consisting of the non-steroidal aromatase inhibitors anastrozole, fadrozole (including the (+)-enantiomer thereof), as well as the steroidal aromatase inhibitor exemestane, or, in each case where applicable, a pharmaceutically acceptable salt thereof.

The most preferred non-steroidal aldosterone synthase inhibitor is the (+)-enantiomer of the hydrochloride of fadrozole (U.S. Pat. Nos. 4,617,307 and 4,889,861) of formula

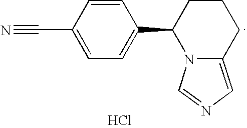

A preferred steroidal aldosterone antagonist is eplerenone of the formula

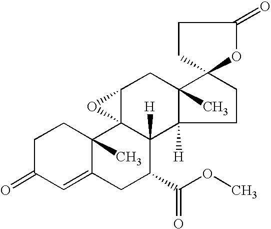

or

spironolactone.

A preferred dual angiotensin converting enzyme/neutral endopetidase (ACE/NEP) inhibitor is, for example, omapatrilate (cf. EP 629627), fasidotril or fasidotrilate, or, if appropriable, a pharmaceutically acceptable salt thereof.

A preferred endothelin antagonist is, for example, bosentan (cf. EP 526708 A), furthermore, tezosentan (cf. WO 96/19459), or in each case, a pharmaceutically acceptable salt thereof.

A renin inhibitor is, for example, a non-peptidic renin inhibitor such as the compound of formula

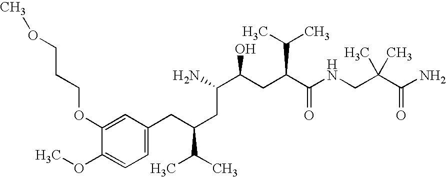

chemically defined as 2(S),4(S),5(S),7(S)—N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide. This representative is specifically disclosed in EP 678503 A. Especially preferred is the hemi-fumarate salt thereof.

A diuretic is, for example, a thiazide derivative selected from the group consisting of chlorothiazide, hydrochlorothiazide, methylclothiazide, and chlorothalidon. The most preferred is hydrochlorothiazide.

An ApoA-I mimic is, for example, D4F peptide, especially of formula D-W-F-K-A-F-Y-D-K-V-A-E-K-F-K-E-A-F

Preferably, the jointly therapeutically effective amounts of the active agents according to the combination of the present invention can be administered simultaneously or sequentially in any order, separately or in a fixed combination.

The structure of the active agents identified by generic or tradenames may be taken from the actual edition of the standard compendium “The Merck Index” or from databases, e.g. IMS LifeCycle (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

Furthermore, the combinations as described above can be administered to a subject via simultaneous, separate or sequential administration (use). Simultaneous administration (use) can take place in the form of one fixed combination with two or more active ingredients, or by simultaneously administering two or more compounds that are formulated independently. Sequential administration (use) preferably means administration of one (or more) compounds or active ingredients of a combination at one time point, other compounds or active ingredients at a different time point, that is, in a chronically staggered manner, preferably such that the combination shows more efficiency than the single compounds administered independently (especially showing synergism). Separate administration (use) preferably means administration of the compounds or active ingredients of the combination independently of each other at different time points, preferably meaning that two compounds are administered such that no overlap of measurable blood levels of both compounds are present in an overlapping manner (at the same time).

Also combinations of two or more of sequential, separate and simultaneous administrations are possible, preferably such that the combination compound-drugs show a joint therapeutic effect that exceeds the effect found when the combination compound-drugs are used independently at time intervals so large that no mutual effect on their therapeutic efficiency can be found, a synergistic effect being especially preferred.

Additionally, the present invention provides:
a pharmaceutical composition or combination of the present invention for use as a medicament;
the use of a pharmaceutical composition or combination of the present invention for the delay of progression and/or treatment of a disorder or disease mediated by CETP or responsive to the inhibition of CETP.
the use of a pharmaceutical composition or combination of the present invention for the delay of progression and/or treatment of a disorder or disease selected from hyperlipidemia, arteriosclerosis, atherosclerosis, peripheral vascular disease, dyslipidemia, hyperbetalipoproteinemia, hypoalphalipoproteinemia, hypercholesterolemia, hypertriglyceridemia, familial hypercholesterolemia, cardiovascular disorder, coronary heart disease, coronary artery disease, coronary vascular disease, angina, ischemia, heart ischemia, thrombosis, cardiac infarction such as myocardial infarction, stroke, peripheral vascular disease, reperfusion injury, angioplasty restenosis, hypertension, congestive heart failure, diabetes such as type II diabetes mellitus, diabetic vascular complications, obesity or endotoxemia etc.

The pharmaceutical composition or combination of the present invention can be in unit dosage of about 1-1000 mg of active ingredients for a subject of about 50-70 kg, preferably about 5-500 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., preferably aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, preferably between about 1-100 mg/kg.

The CETP inhibitory effect of the compounds of the present invention can be determined by using the test models or assays known in the art. For example, EP1115695B1 describes both the in vitro and in vivo CETP activity assays, the contents of which are hereby incorporated by reference. In particular, the following assays are used.

(1) CETP in vitro Assay:

CETP Activity Kit (#RB-RPAK) was purchased from Roar Biochemical, Inc. (New York, N.Y., USA). To each well of a 96-well NBS half-area plate (costar #3686), 1.2 ng/well of the donor solution, 1 μL of the acceptor solution and 5 μL compound solution diluted in 100% DMSO are added in a 38 μL of buffer containing 10 mM Tris, 150 mM NaCl and 2 mM EDTA, pH 7.4. Then, the plate is sealed with Themowell™ Sealers (costar #6524) and followed by a mixing on a plate shaker by MICROPLATE MIXER MPX-96 (IWAKI) at power 3 for 10 sec at room temperature. After 10-min incubation at 37° C., the reaction is started by adding 5 μL of rhCETP solution (Cardiovascular Target, New York, N.Y., USA) and mixed on the plate shaker for 10 sec, then the fluorescence intensity at 0 min is measured by a ARVO SX (Perkin Elmerr, USA) at excitation wavelength of 465 nm and emission wavelength of 535 nm. After 120 min-incubation at 37° C., fluorescence intensity is measured again. The inhibition of rhCETP activity by a compound is calculated by the following calculation. Inhibition %={1−(F120−F0)/(f120−f0)}×100 F: measured fluorescence intensity with compound at 0 or 120 min. f: measured fluorescence intensity of without compound at 0 or 120 min.

The IC₅₀ values are determined from the dose-effect curve by Origin software. IC₅₀ values, especially from about 0.1 nM to about 50 μM, are determined for the compounds of the present invention or a pharmaceutically acceptable salt thereof.

(2) Effects on Plasma HDL Levels in Hamster:

Effects of compounds on HDL-cholesterol level in hamsters are investigated by the method reported previously with some modifications (Eur, J. Phamacol, 466 (2003) 147-154). In brief, male Syrian hamsters (10-11 week-old age, SLC, Shizuoka, Japan) are fed a high cholesterol diet for two weeks. Then, the animals are dosed singly with the compound suspended with carboxyl methyl cellulose solution. HDL-cholesterol levels are measured by using commercially available kit (Wako Pure Chemical, Japan) after the precipitation of apolipoprotein B (apoB)-containing lipoproteins with 13% polyethylene glycol 6000.

(3) Preparation of Human Pro-Apolipoprotein Al (pro-apoAl)

The cDNA of human pro-apoAl (NCBI accession number: NM_{—}000039) is cloned from human liver Quick-Clone™ cDNA (Clontech, Calif.) and inserted to a pET28a vector (Novagen, Germany) for bacterial expression. Expressed protein as a fusion protein with 6×His-tag at N-terminus in BL-21 Gold (DE3) (Strategene, Calif.) is purified using HiTrap Chelating (GE Healthcare, Conn.).

(4) Preparation of Donor Microemulsion

Pro-apoAl containing microemulsion as a donor particle is prepared following previous reports (J. Biol. Chem., 280:14918-22). Glyceryl trioleate (62.5 ng, Sigma, MO), 3-sn-phosphatidylcholine (583 ng, Wako Pure Chemical Industries, Japan), and cholesteryl BODIPY® FL C₁₂ (250 ng, Invitrogen, Calif.) are dissolved in 1 mL of chloroform. The solution is evaporated, then residual solvent is removed in vacuum for more than 1 hr. The dried lipid mixture is dissolved in 500 μL of the assay buffer (50 mM Tris-HCl (pH7.4) containing 150 mM NaCl and 2 mM EDTA) and sonicated at 50° C. with a microtip (MICROSON™ ULTRASONIC CELL DISRUPTOR, Misonix, Farmingdale, N.Y.) at output power 006 for 2 min. After sonication, the solution is cooled to 40° C., added to 100 μg of human pro-apoAl, and sonicated at output power 004 for 5 min at 40° C. The solution, BODIPY-CE microemulsion as a donor molecule is stored at 4° C. after filtration through a 0.45 μm PVDF filter.

(5) In vitro CETP Activity Assay in Human Plasma

Human EDTA plasma samples from healthy men are purchased from New Drug Development Research Center, Inc. Donor solution is prepared by a dilution of donor microemulsion with assay buffer. Human plasma (50 μL), assay buffer (35 μL) and test compound dissolved in dimethylsulfoxide (1 μL) are added to each well of 96 well half area black flat bottom plate. The reaction is started by the addition of donor solution (14 μL) into each well. Fluorescence intensities are measured every 30 min at 37° C. with excitation wave length of 485 nm and emission wavelength of 535 nm. The CETP activity (Fl/min) is defined as the changes of fluorescence intensity from 30 to 90 min. The IC₅₀ value is obtained by the logistic equation (Y=Bottom+(Top-Bottom)/(1+(x/IC₅₀)̂Hill slope) using Origin software, version 7.5 SR3. The compounds of formula I exhibit inhibitory activity with an IC50 value in the range from approximately from 0.001 to 100 μM, especially from 0.01 to 10 μM.

The compounds of the present invention or a pharmaceutically acceptable salt thereof have superior CETP inhibitory activity in mammals (e.g., human, monkey, bovine, horse, dog, cat, rabbit, rat, mouse and the like), and can be used as CETP activity inhibitors. In addition, utilizing the superior CETP inhibitory activity of a compound of the present invention or a pharmaceutically acceptable salt thereof, the compounds of the present invention are useful as pharmaceutical agents effective for the prophylaxis or treatment of or delay progression to overt to diseases in which CETP is involved (e.g., hyperlipidemia, arteriosclerosis, atherosclerosis, peripheral vascular disease, dyslipidemia, hyperbetalipoproteinemia, hypoalphalipoproteinemia, hypercholesterolemia, hypertriglyceridemia, familial hypercholesterolemia, cardiovascular disorder, coronary heart disease, coronary artery disease, coronary vascular disease, angina, ischemia, heart ischemia, thrombosis, cardiac infarction such as myocardial infarction, stroke, peripheral vascular disease, reperfusion injury, angioplasty restenosis, hypertension, congestive heart failure, diabetes such as type II diabetes mellitus, diabetic vascular complications, obesity or endotoxemia etc.), particularly as prophylactic or therapeutic agents for hyperlipidemia or arteriosclerotic diseases.

Abbreviations

Ac: Acetyl

dba:dibenzylidenacetone

DMAP: N,N-dimethylaminopyridine

DME: dimethoxyethane

DMF: N,N-dimethylformamide

dppf: 1,1-bis(diphenylphosphino)ferrocene

ESI: electrospray ionization

EtOAc, AcOEt: ethyl acetate

h: hours

HPLC: high pressure liquid chromatography

IPA: 2-propanol

iPr: isopropyl

LC: liquid chromatography

LHMDS: lithium hexamethyldisilamide

min: minutes

MS: mass spectrometry

NMR: nuclear magnetic resonance

sat.: saturated

THF: tetrahydrofuran

tol: tolyl

UPLC: Ultra performance liquid chromatography

### EXAMPLES

The following examples are intended to illustrate the invention and are not to be construed as being limitations thereon. Temperatures are given in degrees centrigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art. The compounds in the following examples have been found to have IC₅₀ values in the range of about 0.1 nM to about 100,0.00 nM for CETP.

General UPLC Condition

Column: Waters ACQUITY UPLC BEH C18, 1.7 μM

Mobile phase: CH₃CN/H₂O (0.1% TFA)

Preparation of the Starting Materials

### Example a

### Preparation of 3-bromo-2-chloro-5-trifluoromethylpyridine

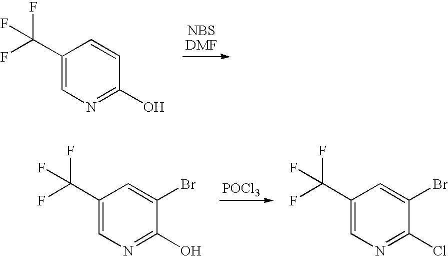

Step 1:

N-bromosuccinimide (NBS, 39.00 g, 0.22 mol) is added portionwise to a solution of 5-(trifluoromethyl)pyridin-2-ol (30.00 g, 0.18 mol) in DMF (180 mL), and the resulting mixture is stirred for 2 hours. The mixture is poured into water (1200 mL) and the precipitate is collected by filtration. The crystal is dried in vacuo to give the product as a white solid (1st crystal: 28.10 g). The filtrate is extracted with EtOAc, and the organic layer is concentrated. The residue is poured into water and the precipitate is collected by filtration. The crystal is dried in vacuo to give 3-bromo-5-(trifluoromethyl)pyridin-2-ol as a yellow solid.

¹H-NMR (400 MHz, CDCl₃), δ (ppm): 7.86 (d, 1H), 8.02 (d, 1H), 13.17 (br, 1H).

Step 2:

A mixture of 3-bromo-5-(trifluoromethyl)pyridin-2-ol (37.75 g, 0.16 mol) and phosphorus(III) oxychloride (POCl₃; 75 mL) is stirred at 100° C. for 5 hours. After cooling to room temperature, the mixture is poured into ice-water, and extracted with CH₂Cl₂ twice. The combined organic layer is washed with NaHCO₃ aq., brine, dried over MgSO₄, filtered and concentrated in vacuo. The crude mixture is purified by flash column chromatography to give 3-bromo-2-chloro-5-trifluoromethylpyridine as a white solid.

¹H-NMR (400 MHz, CDCl₃), δ (ppm): 8.17 (m, 1H), 8.62 (d, 1H).

### Example b

### Preparation of N-[3,5-bis(trifluoromethyl)benzyl]-N-{2-[2-(tetrahydropyran-2-yloxy)ethyl]-2H-tetrazol-5-yl}amine

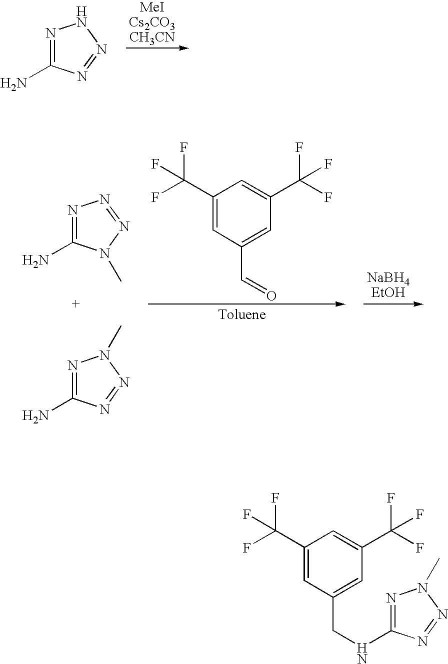

A mixture of 5-aminotetrazole (24.4 g, 0.29 mol), methyliodide (48.8 g, 0.34 mol), and Cs₂CO₃ (112.0 g, 0.34 mol) in acetonitrile (700 mL) is stirred and refluxed for 7 hours. The mixture is cooled to 50° C. and filtrated. The resulting filtrate is concentrated to give the mixture of 5-amino-2-methyltetrazole and 5-amino-1-methyltetrazole.

A mixture of the crude product and 3,5-bis(trifluoromethyl)benzaldehyde (43.0 g, 0.18 mol) in toluene (600 mL) is stirred and refluxed for 45 min. After cooling to room temperature, the resulting mixture is concentrated. NaBH₄ (8.12 g, 0.22 mol) is added portionwise slowly to EtOH (500 mL) solution of the resulting residue, and the mixture is stirred at room temperature for 4 hours. After addition of sat. NH₄Cl aq. and water, the mixture is extracted with ethyl acetate. The combined organic layer is washed with brine, dried over magnesium sulfate, filtered and concentrated. The crude product is purified by crystallization (50 mL of i-PrOH:H₂O. 3:7) to give [3,5-bis(trifluoromethyl)phenylmethyl](2-methyl-2H-tetrazol-5-yl)amine.

### Example c

### Preparation of [3,5-bis(trifluoromethyl)benzyl](2-chloro-5-trifluoromethylpyridin-3-ylmethyl)(2-methyl-2H-tetrazol-5-yl)amine

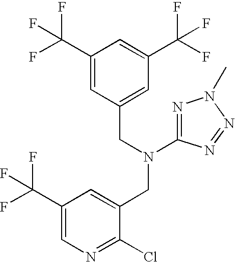

n-BuLi (1.57M solution in hexane; 64 mL, 0.10 mol) is added dropwise to a solution of 3-bromo-2-chloro-5-trifluoromethylpyridine (20.00 g, 0.077 mol), DMF (7.72 mL, 0.10 mol) in toluene (400 mL) at −65° C. After stirring at the same temperature for 30 min, the mixture is quenched by addition of 1N HCl and extracted with ethyl acetate. The organic layer is washed with water, brine, dried over magnesium sulfate, filtered and concentrated to give crude 2-chloro-5-trifluoromethylpyridine-3-carbardehyde.

To a solution of crude 2-chloro-5-trifluoromethylpyridine-3-carbardehyde in ethanol (60 mL), sodium tetraborohydride (2.90 g, 0.077 mol) is added portionwise and stirred for 30 min at room temperature. After adding sat. ammonium chloride solution, the mixture is extracted with ethyl acetate. The organic layer is washed with sat. ammonium chloride solution, brine, dried over magnesium sulfate, filtered and concentrated. The residue is purified by silica gel column chromatography to give 2-chloro-5-trifluoromethylpyridin-3-ylmethanol.

Methanesulfonyl chloride (3.4 mL, 0.044 mol) and N,N-diisopropylethylamine (7.8 mL, 0.045 mol) are added dropwise to a solution of 2-chloro-5-trifluoromethylpyridin-3-ylmethanol (3.72 g g, 0.018 mol) in toluene (90 mL) at 0° C. and the mixture is stirred for 12 hours at room temperature. The mixture is diluted with water, and sat. NaHCO₃ aqueous solution, the mixture is extracted with ethyl acetate. The combined organic layer is washed with brine, dried over magnesium sulfate, filtered and concentrated to give crude 2-chloro-3-chloromethyl-5-trifluoromethylpyridine.

Lithium bis(trimethylsilyl)amide (1.0M in THF; 25.2 mL, 0.025 mol) is added dropwise to a solution of N-[3,5-bis(trifluoromethyl)phenylmethyl]-N-(2-methyl-2H-tetrazol-5-yl)amine (7.15 g, 0.022 mmol) in THF (60 mL) and the mixture is stirred for 30 min at room temperature. This solution is added dropwise to a solution of crude 2-chloro-3-chloromethyl-5-trifluoromethylpyridine in DMF (60 mL) at −40° C. and the mixture is stirred for 3 hours at same temperature. After warming up to room temperature, the mixture is quenched by addition of sat. ammonium chloride solution and extracted with ethyl acetate twice. The combined organic layer is washed with water, brine, dried over magnesium sulfate, filtered and concentrated. The residue is purified by silica gel column chromatography to give 3,5-bis(trifluoromethyl)benzyl](2-chloro-5-trifluoromethylpyridin-3-ylmethyl)(2-methyl-2H-tetrazol-5-yl)amine.

¹H-NMR (400 MHz, CDCl3): 4.21 (s, 3H), 4.81 (s, 2H), 4.87 (s, 2H), 7.71 (s, 2H), 7.72-7.79 (m, 1H), 7.79 (s, 1H), 8.56 (s, 1H).

### Example d

### Preparation of 3-{[(3,5-Bis-trifluoromethyl-benzyl)-(2-methyl-2H-tetrazol-5-yl)-amino]-methyl}-5-trifluoromethyl-pyridine-2-carbonitrile

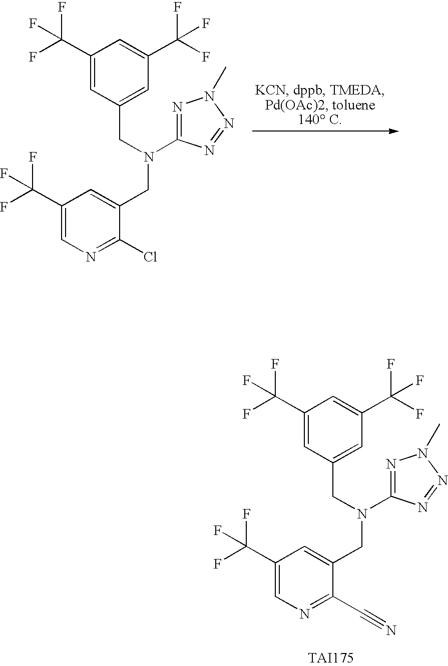

To a solution of [3,5-bis(trifluoromethyl)benzyl](2-chloro-5-trifluoromethylpyridin-3-ylmethyl)(2-methyl-2H-tetrazol-5-yl)amine (775 mg, 1.5 mmol) in toluene (10 mL), Pottasium cyanide (292 mg, 4.5 mmol), diphenylphosphinobutane (255 mg, 0.6 mmol), palladium acetate (67 mg, 0.3 mmol) and N,N,N′,N′-tetramethyl-ethane-1,2-diamine (1.2 mL, 7.5 mmol) are added at room temperature, and stirred at 130° C. for 2 hours. After cooling to room temperature, the mixture is extracted with ethyl acetate. The combined organic layer is washed with brine, dried over magnesium sulfate, filtrated and concentrated. The residue is purified by silica gel column chromatography to give 3-{[(3,5-Bis-trifluoromethyl-benzyl)-(2-methyl-2H-tetrazol-5-yl)-amino]-methyl}-5-trifluoromethyl-pyridine-2-carbonitrile.

¹H-NMR (400 MHz, CDCl3): 4.20 (s, 3H), 4.93 (s, 4H), 7.71 (s, 2H), 7.79 (s, 1H), 8.04 (s, 1H), 8.84 (s, 1H).

### Example e

### Preparation of 3-{[(3,5-Bis-trifluoromethyl-benzyl)-(2-methyl-2H-tetrazol-5-yl)-amino]-methyl}-5-trifluoromethyl-pyridine-2-carboxylic acid

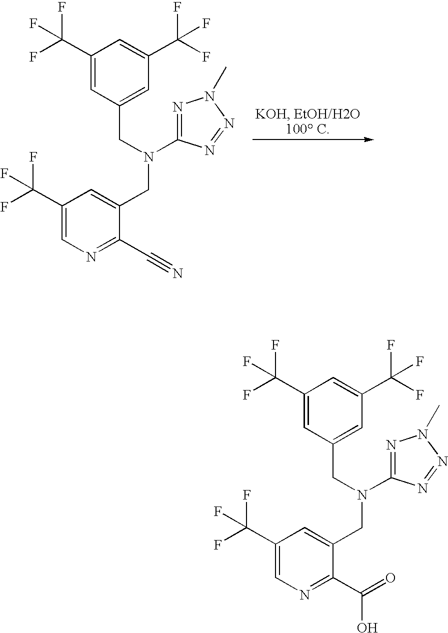

To a solution of 3-{[(3,5-Bis-trifluoromethyl-benzyl)-(2-methyl-2H-tetrazol-5-yl)-amino]-methyl}-5-trifluoromethyl-pyridine-2-carbonitrile (69 mg, 0.14 mmol) in ethanol (2.5 mL)/H2O (0.5 mL), pottasium hydroxide (76 mg, 1.4 mmol) is added at room temperature, and stirred at 100° C. for 2 hours. After adding 1 mol/L HCl aq. at 0° C., the mixture is extracted with ethyl acetate. The combined organic layer is washed with brine, dried over magnesium sulfate, filtrated, and evaporated to afford the title compound as a white amorphous solid.

¹H-NMR (400 MHz, CDCl3): 4.18 (s, 3H), 4.92 (s, 2H), 5.35 (s, 2H), 7.71 (s, 2H), 7.76 (s, 1H), 8.02 (s, 1H), 8.75 (s, 1H). ES-MS: M+H=528; UPLC: RT=3.96 min.

### Example f

### Preparation of 3-{[(3,5-bis-trifluoromethyl-benzyl)-(2-methyl-2H-tetrazol-5-yl)-amino]-methyl}-5-trifluoromethyl-pyridine-2-carboxylic acid methoxy-methyl-amide

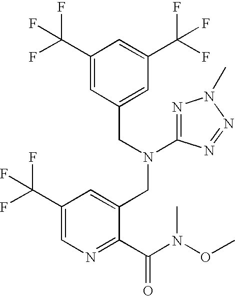

To a stirred solution of 3-{[(3,5-Bis-trifluoromethyl-benzyl)-(2-methyl-2H-tetrazol-5-yl)-amino]-methyl}-5-trifluoromethyl-pyridine-2-carboxylic acid (685 mg, 1.3 mmol) in DMF (12 mL), N,O-dimethylamine hydrochloride (506 mg, 5.2 mmol) is added, and then WSCD (1.2 g, 5.2 mmol) and HOAt (809 mg, 5.2 mmol) are added. After stirring at room temperature over night, the reaction mixture is diluted with saturated NaHCO3 solution, and the aqueous layer is extracted with EtOAc. The combined organic layer is washed with brine, dried over magnesium sulfate, filtrated and concentrated in vacuo. The residue is purified by silica gel column chromatography (AcOEt/hexane=1/3) to afford 720 mg of the title compound.

¹H-NMR (400 MHz, CDCl3): 3.32 (s, 3H), 3.60 (s, 3H), 4.20 (s, 3H), 4.76 (s, 4H), 7.70 (s, 2H), 7.75 (s, 1H), 7.88 (s, 1H), 8.77 (s, 1H). ES-MS: M+=571; UPLC: RT=2.11 min.

### Example g

### Preparation of 1-(3-{[(3,5-bis-trifluoromethyl-benzyl)-(2-methyl-2H-tetrazol-5-yl)-amino]-methyl}-5-trifluoromethyl-pyridin-2-yl)-propan-1-one

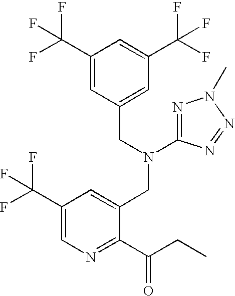

To a stirred solution of 3-{[(3,5-bis-trifluoromethyl-benzyl)-(2-methyl-2H-tetrazol-5-yl)-amino]-methyl}-5-trifluoromethyl-pyridine-2-carboxylic acid methoxy-methyl-amide (420 mg, 0.77 mmol) in THF (8 mL), EtMgBr in 0.97M THF solution (1.58 mL, 1.54 mmol) is added at 0° C. The reaction mixture is stirred at 0° C. for 30 min before adding sat. NH4Cl aq. The aqueous layer is extracted with EtOAc. The combined organic layer is washed with sat. NH4Cl aq. and brine, dried over magnesium sulfate, filtrated and concentrated in vacuo. The residue is purified by silica gel column chromatography (AcOEt/hexane=1/4) to afford 1-(3-{[(3,5-Bis-trifluoromethyl-benzyl)-(2-methyl-2H-tetrazol-5-yl)-amino]-methyl}-5-trifluoromethyl-pyridin-2-yl)-propan-1-one.

ES-MS: M+=541; UPLC: RT=2.31 min.

### Example h

### Preparation of (3-{[(3,5-bis-trifluoromethyl-benzyl)-(2-methyl-2H-tetrazol-5-yl)-amino]-methyl}-5-trifluoromethyl-pyridin-2-yl)-cyclohexyl-methanone

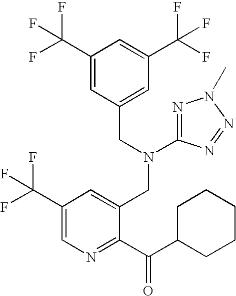

To a stirred solution of 3-{[(3,5-bis-trifluoromethyl-benzyl)-(2-methyl-2H-tetrazol-5-yl)-amino]-methyl}-5-trifluoromethyl-pyridine-2-carboxylic acid methoxy-methyl-amide (230 mg, 0.40 mmol) in THF (5 mL), c-HexMgBr in 1.0 M THF solution (1.58 mL, 1.54 mmol) is added at 0° C. The reaction mixture is stirred at 0° C. for 30 min before adding sat. NH4Cl aq. The aqueous layer is extracted with EtOAc. The combined organic layers are washed with sat. NH4Cl aq., and brine, dried over magnesium sulfate, filtrated and concentrated in vacuo. The residue is purified by silica gel column chromatography (AcOEt/hexane=1/4) to afford the title compound.

1.21-1.29 (m, 1H), 1.33-1.40 (m, 4H), 1.70-1.75 (m, 1H), 1.78-1.84 (m, 4H), 3.70-3.75 (m, 1H), 4.17 (s, 3H), 4.85 (s, 2H), 4.99 (s, 2H), 7.70 (s, 2H), 7.76 (s, 1H), 7.86 (s, 1H), 8.78 (s, 1H). ES-MS: M+H=595; UPLC: RT=2.47 min.

### Example i

### Preparation of (E)-3-(3-{[(3,5-bis-trifluoromethyl-benzyl)-(2-methyl-2H-tetrazol-5-yl)-amino]-methyl}-5-trifluoromethyl-pyridin-2-yl)-acrylic acid methyl ester

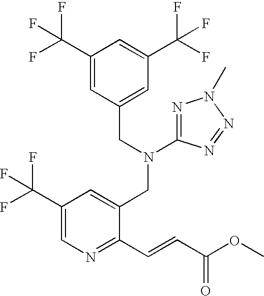

To a solution of [3,5-bis(trifluoromethyl)benzyl}(2-chloro-5-trifluoromethylpyridin-3-ylmethyl)(2-methyl-2H-tetrazol-5-yl)amine (560 mg, 1.08 mmol) in DMF (10 mL), methylacrylate (194 μL, 2.16 mmol), tetrabutylammonium bromide (1.0 g, 3.24 mmol), palladium acetate (24 mg, 0.11 mmol), triethylamine (0.45 mL, 3.24 mmol) and diphehylphosphino ferrocene (180 mg, 0.32 mmol) are added. After stirring at 120° C. for 4 h under nitrogen atmosphere, the reaction mixture is diluted with H2O, and the aqueous layer is extracted with EtOAc. The combined organic layers are washed with brine, dried over magnesium sulfate, filtrated and concentrated in vacuo. The residue is purified by silica gel column chromatography (AcOEt/hexane=1/4) to afford the title compound.

¹H-NMR (400 MHz, CDCl3): 3.79 (s, 3H), 4.23 (s, 3H), 4.78 (s, 2H), 4.89 (s, 2H), 7.09 (d, 1H), 7.59 (s, 2H), 7.70 (s, 1H), 7.75 (s, 1H), 7.85 (d, 1H), 8.75 (s, 1H). ES-MS: M+H=569; UPLC: RT=2.22 min.

### Example 1

### Preparation of 1-(3-{[(3,5-bis-trifluoromethyl-benzyl)-(2-methyl-2H-tetrazol-5-yl)-amino]-methyl}-5-trifluoromethyl-pyridin-2-yl)-propan-1-ol

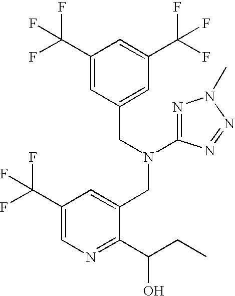

To a stirred solution of 1-(3-{[(3,5-Bis-trifluoromethyl-benzyl)-(2-methyl-2H-tetrazol-5-yl)-amino]-methyl}-5-trifluoromethyl-pyridin-2-yl)-propan-1-one (140 mg, 0.26 mmol) in EtOH (4 mL), sodium borohydride (1.58 mL, 1.54 mmol) is added at 0° C. The reaction mixture is stirred at 0° C. for 30 min before adding sat. NH4Cl aq. The aqueous layer is extracted with EtOAc. The combined organic layers are washed with sat. NH4Cl aq., and brine, dried over magnesium sulfate, filtrated and concentrated in vacuo to afford the title compound.

¹H-NMR (400 MHz, CDCl3): 0.95 (t, 3H), 1.55 (m, 1H), 1.74 (m, 1H), 4.07 (d, 1H), 4.20 (s, 3H), 4.70-4.79 (m, 4H), 7.63 (s, 1H), 7.66 (s, 2H), 7.79 (s, 1H), 8.73 (s, 1H). ES-MS: M+H=543; UPLC: RT=2.13 min.

### Example 2

### Preparation of (3,5-Bis-trifluoromethyl-benzyl)-[2-(1-cyclohexylamino-propyl)-5-trifluoromethyl-pyridin-3-ylmethyl]-(2-methyl-2H-tetrazol-5-yl)-amine

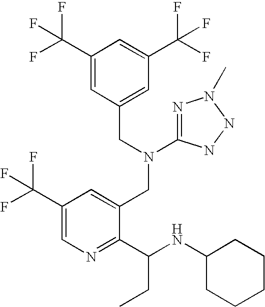

To a stirred solution of 1-(3-{[(3,5-bis-trifluoromethyl-benzyl)-(2-methyl-2H-tetrazol-5-yl)-amino]-methyl}-5-trifluoromethyl-pyridin-2-yl)-propan-1-ol (35 mg, 0.13 mmol) in toluene, methanesulfonyl chloride (20 uL, 0.26 mmol) and diisopropylethylamine (45 uL, 0.26 mmol) are added. The reaction mixture is stirred at room temperature for 1 h. The aqueous layer is extracted with EtOAc. The combined organic layers are washed with sat. NaHCO3 aq., and brine, dried over magnesium sulfate, filtrated and concentrated in vacuo.

To a solution of the resulting mixture in DMSO, cyclohexylamine (64 mg, 0.65 mmol) and diisopropylethylamine (110 uL, 0.65 mmol) are added. After stirring at 50° C., the reaction mixture is diluted with H2O, and the aqueous layer is extracted with EtOAc. The combined organic layers are washed with brine, dried over magnesium sulfate, filtrated and concentrated in vacuo. The residue is purified by silica gel column chromatography (AcOEt/hexane=1/3) to afford 18 mg of the title compound.

¹H-NMR (400 MHz, CDCl3): 0.78 (t, 3H), 1.05-1.09 (m, 5H), 1.57-1.76 (m, 7H), 2.04-2.11 (m, 1H), 3.95 (t, 1H), 4.20 (s, 3H), 4.77 (q, 2H), 4.84 (s, 2H), 7.52 (s, 1H), 7.68 (s, 2H), 7.80 (s, 1H), 8.75 (s, 1H). ES-MS: M+H=624; UPLC: RT=1.98 min.

### Example 3

The following compounds are prepared according to the method of example 1 by using appropriate reagents and conditions.

### Example 3-1

### Preparation of (3,5-Bis-trifluoromethyl-benzyl)-(2-methyl-2H-tetrazol-5-yl)-[2-(1-piperidin-1-yl-propyl)-5-trifluoromethyl-pyridin-3-ylmethyl]-amine

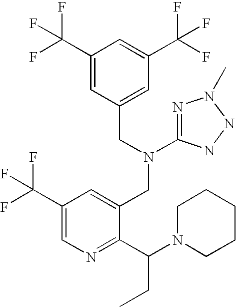

¹H-NMR (400 MHz, CDCl3): 0.73 (t, 3H), 1.24-1.30 (m, 6H), 1.69-1.76 (m, 1H), 2.15-2.23 (m, 1H), 2.31-2.35 (m, 2H), 2.44-2.47 (m, 2H), 4.20 (s, 3H), 4.76 (d, 2H), 4.86 (d, 2H), 5.00 (d, 2H), 5.04 (d, 2H), 7.56 (s, 1H), 7.67 (s, 2H), 7.78 (s, 1H), 8.70 (s, 1H). ES-MS: M+H=610; UPLC: RT=1.94 min.

### Example 3-2

### Preparation of (3,5-bis-trifluoromethyl-benzyl)-{2-[1-(cyclohexyl-methyl-amino)-propyl]-5-trifluoromethyl-pyridin-3-ylmethyl}-(2-methyl-2H-tetrazol-5-yl)-amine

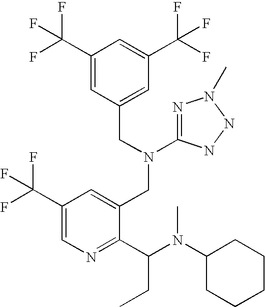

¹H-NMR (400 MHz, CDCl3): 0.73 (t, 3H), 1.24-1.30 (m, 6H), 1.69-1.76 (m, 1H), 2.15-2.23 (m, 1H), 2.31-2.35 (m, 2H), 2.44-2.47 (m, 2H), 4.20 (s, 3H), 4.76 (d, 2H), 4.86 (d, 2H), 5.00 (d, 2H), 5.04 (d, 2H), 7.56 (s, 1H), 7.67 (s, 2H), 7.78 (s, 1H), 8.70 (s, 1H). ES-MS: M+H=610; UPLC: RT=1.94 min.

### Example 3-3

### Preparation of (3,5-bis-trifluoromethyl-benzyl)-(2-methyl-2H-tetrazol-5-yl)-[2-(1-pyrrolidin-1-yl-propyl)-5-trifluoromethyl-pyridin-3-ylmethyl]-amine

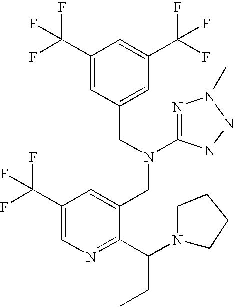

¹H-NMR (400 MHz, CDCl3): 0.66 (t, 3H), 1.66 (bs, 4H), 1.91-1.96 (m, 1H), 2.27-2.35 (m, 2H), 2.50-2.56 (m, 2H), 4.54-4.57 (m, 1H), 4.20 (s, 3H), 4.77 (s, 2H), 4.89 (d, 2H), 5.05 (d, 2H), 7.59 (s, 1H), 7.69 (s, 2H), 7.80 (s, 1H), 8.77 (s, 1H). ES-MS: M+H=596

### Example 3-4

### Preparation of (3,5-bis-trifluoromethyl-benzyl)-[2-(1-cyclopentylamino-propyl)-5-trifluoromethyl-pyridin-3-ylmethyl]-(2-methyl-2H-tetrazol-5-yl)-amine

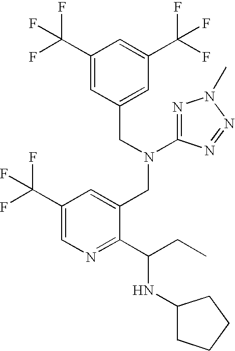

¹H-NMR (400 MHz, CDCl3): 0.78 (t, 3H), 1.16-1.42 (m, 6H), 1.62 (bs, 4H), 2.69-2.75 (m, 1H), 3.82-3.88 (m, 1H), 4.20 (s, 3H), 4.77 (s, 2H), 4.82 (s, 2H), 7.53 (s, 1H), 7.68 (s, 2H), 7.79 (s, 1H), 8.76 (s, 1H). ES-MS: M+H=610

### Example 3-5

### Preparation of (3,5-bis-trifluoromethyl-benzyl)-{2-[1-((R)-3-dimethylamino-pyrrolidin-1-yl)-propyl]-5-trifluoromethyl-pyridin-3-ylmethyl}-(2-methyl-2H-tetrazol-5-yl)-amine

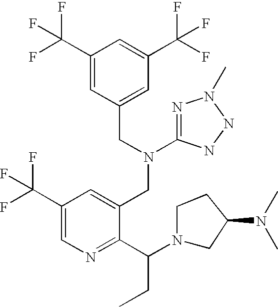

¹H-NMR (400 MHz, CDCl3): 0.64-0.69 (m, 3H), 1.57-2.02 (m, 6H), 2.10 (s, 3H), 2.14 (s, 3H), 2.22 (t, 0.5H), 2.28 (t, 0.5H), 2.81-2.85 (m, 0.5H), 3.00-3.04 (m, 0.5H), 3.62-3.67 (m, 1H), 4.19 (s, 1.5H), 4.20 (s, 1.5H), 4.73 (d, 1H), 4.80 (d, 1H), 4.89 (d, 1H), 5.04 (d, 1H), 7.59 (s, 1H), 7.68 (s, 2H), 7.79 (s, 1H), 8.75(s, 1H). ES-MS: M+H=639

### Example 3-6

### Preparation of (3,5-bis-trifluoromethyl-benzyl)-{2-[1-((S)-3-dimethylamino-pyrrolidin-1-yl)-propyl]-5-trifluoromethyl-pyridin-3-ylmethyl}-(2-methyl-2H-tetrazol-5-yl)-amine

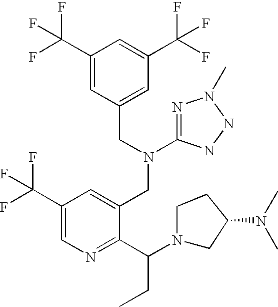

¹H-NMR (400 MHz, CDCl3): 0.64-0.69 (m, 3H), 1.57-2.02 (m, 6H), 2.10 (s, 3H), 2.14 (s, 3H), 2.22 (t, 0.5H), 2.28 (t, 0.5H), 2.81-2.85 (m, 0.5H), 3.00-3.04 (m, 0.5H), 3.62-3.67 (m, 1H), 4.19 (s, 1.5H), 4.20 (s, 1.5H), 4.73 (d, 1H), 4.80 (d, 1H), 4.89 (d, 1H), 5.04 (d, 1H), 7.59 (s, 1H), 7.68 (s, 2H), 7.79 (s, 1H), 8.75(s, 1H). ES-MS: M+H=639

### Example 4

### Preparation of (3,5-bis-trifluoromethyl-benzyl)-[2-(cyclohexyl-methoxy-methyl)-5-trifluoromethyl-pyridin-3-ylmethyl]-(2-methyl-2H-tetrazol-5-yl)-amine

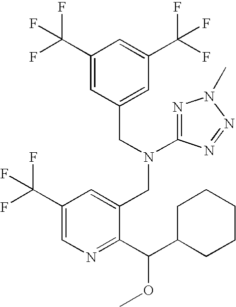

To a stirred solution of (3-{[(3,5-bis-trifluoromethyl-benzyl)-(2-methyl-2H-tetrazol-5-yl)-amino]-methyl}-5-trifluoromethyl-pyridin-2-yl)-cyclohexyl-methanone (47 mg, 0.08 mmol) in EtOH (2 mL), sodium borohydride (6 mg, 0.16 mmol) is added at 0° C. The reaction mixture is stirred at 0° C. for 30 min before adding sat. NH4Cl aq. The aqueous layer is extracted with EtOAc. The combined organic layers are washed with sat. NH4Cl aq., and brine, dried over magnesium sulfate, filtrated and concentrated in vacuo.

To a solution of the resulting mixture in DMSO, NaH (4 mg, 0.09 mmol) is added. After stirring at room temperature for 10 min, methyl iodine (6 μL, 0.09 mmol) is added. the reaction mixture is diluted with H2O, and the aqueous layer is extracted with EtOAc. The combined organic layers are washed with brine, dried over magnesium sulfate, filtrated and concentrated in vacuo. The residue is purified by silica gel column chromatography (AcOEt/hexane=1/3) to afford the title compound.

### Example 5

### Preparation of 3-(3-{[(3,5-bis-trifluoromethyl-benzyl)-(2-methyl-2H-tetrazol-5-yl)-amino]-methyl}-5-trifluoromethyl-pyridin-2-yl)-3-methylamino-propionic acid methyl ester

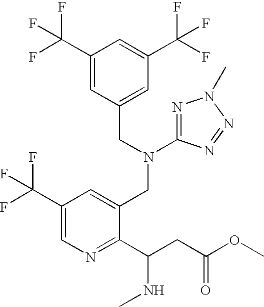

To a mixture of (E)-3-(3-{[(3,5-bis-trifluoromethyl-benzyl)-(2-methyl-2H-tetrazol-5-yl)-amino]-methyl}-5-trifluoromethyl-pyridin-2-yl)-acrylic acid methyl ester (50 mg, 0.08 mmol) and methylamine, 2.0 M THF solution (0.5 mL), LiClO4 (10 mg, 1.0 mmol) is added. After stirring at room temperature for overnight, the reaction mixture is concentrated in vacuo. The residue is purified by silica gel column chromatography (AcOEt/hexane=1/1) to afford the title compound.

¹H-NMR (400 MHz, CDCl3): 2.42 (s, 3H), 3.01 (d, 2H), 3.63 (s, 3H), 4.20 (s, 3H),4.66-4.70 (m, 1H), 4.86 (s, 2H), 4.87 (d, 2H), 4.96 (d, 2H), 7.57 (s, 1H), 7.79 (s, 2H), 7.80 (s, 1H), 8.74 (s, 1H). ES-MS: M+H=600; UPLC: RT=1.85 min.

### Example 6

### Preparation of 3-(3-{[(3,5-Bis-trifluoromethyl-benzyl)-(2-methyl-2H-tetrazol-5-yl)-amino]-methyl}-5-trifluoromethyl-pyridin-2-yl)-3-dimethylamino-propionic acid methyl ester

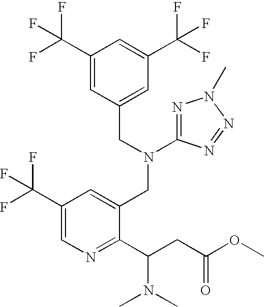

To a mixture of (E)-3-(3-{[(3,5-bis-trifluoromethyl-benzyl)-(2-methyl-2H-tetrazol-5-yl)-amino]-methyl}-5-trifluoromethyl-pyridin-2-yl)-acrylic acid methyl ester (70 mg, 0.12 mmol) and dimethylamine, 2.0 M THF solution (1.0 mL), LiClO4 (26 mg, 0.24 mmol) is added. After stirring at room temperature for 1 week, the reaction mixture is concentrated in vacuo. The residue is purified by silica gel column chromatography (AcOEt/hexane=1/1) to afford the title compound.

¹H-NMR (400 MHz, CDCl3): 2.74 (dd, 1H), 3.38 (dd, 1H), 3.61 (s, 3H), 4.21 (s, 3H), 4.38 (dd, 1H), 4.76 (d, 2H), 4.77 (d, 2H), 4.92 (d, 2H), 5.07 (d, 2H), 7.64 (s, 1H), 7.73 (s, 2H), 7.76 (s, 1H), 8.65 (s, 1H). ES-MS: M+H=614; UPLC: RT=1.87 min.

### Example 7

### Synthesis of [3,5-Bis(trifluoromethyl)phenylmethyl]-[2-(1-cyclohexylaminopropyl)-7-fluoroquinoline-3-ylmethyl]-(2-methyl-2H-tetrazol-5-yl)amine

Step 1:

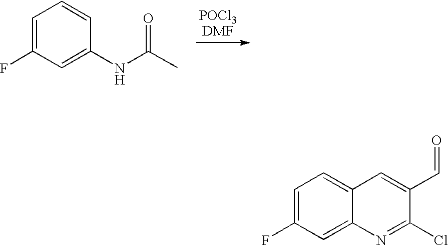

A Vilsmeier reagent prepared from DMF (23 mL) and phosphoryl chloride (78.4 mL) at 0-10° C. is slowly added dropwise to a mixture of 3-fluoroacetanilide (18.5 g, 0.12 mol) and the resulting mixture is stirred at 100° C. for 14 hours. The mixture is poured onto ice-water and extracted with CH₂Cl₂ twice. The combined organic layer is dried, filtered and concentrated. The crystal is collected and washed with CH₂Cl₂ to give 2-chloro-7-fluoroquinoline-3-carbaldehyde as brown powder.

¹H-NMR (400 MHz, CDCl₃), δ (ppm): 7.45 (ddd, 1H), 7.72 (dd, 1H), 8.01 (dd, 1H), 8.76 (s, 1H), 10.55 (s, 1H).

Step 2:

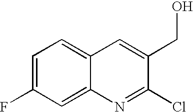

To a solution of crude 2-chloro-7-fluoroquinoline-3-carbaldehyde (2.3 g, 0.011 mmol) in ethanol (50 mL), sodium tetraborohydride (0.5 g, 0.013 mmol) is added portionwise and stirred for 30 min at room temperature. After adding sat. ammonium chloride solution, the mixture is extracted with ethyl acetate. The organic layer is washed with sat. ammonium chloride solution, brine, dried over magnesium sulfate, filtered and concentrated to give crude 2-chloro-7-fluoroquinolin-3-ylmethanol.

¹H-NMR (400 MHz, CDCl₃), δ (ppm): 4.93 (d, 2H), 7.36 (m, 1H), 7.65 (dd, 1H), 7.86 (dd, 1H), 8.29 (s. 1H).

Step 3:

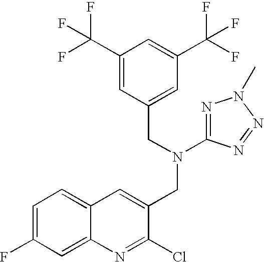

Methanesulfonyl chloride (1.7 mL, 0.022 mmol) and N,N-diisopropylethylamine (3.8 mL, 0.022 mmol) are added dropwise to a solution of crude 2-chloro-7-fluoroquinolin-3-ylmethanol (0.011 mol) in toluene (60 mL) at 0° C. and the mixture is stirred for 30 min at room temperature. The mixture is diluted with water and sat. NaHCO₃ aqueous solution, and then the mixture is extracted with ethyl acetate. The combined organic layer is washed with brine, dried over magnesium sulfate, filtered and concentrated to give crude 2-chloro-3-chloromethyl-7-fluoroquinoline.

Lithium bis(trimethylsilyl)amide (1.0M in THF; 9.5 mL, 0.010 mol) is added dropwise to a solution of N-[3,5-bis(trifluoromethyl)phenylmethyl]-N-(2-methyl-2H-tetrazol-5-yl)amine (2.4 g, 0.007 mmol) in THF (24 mL) and the mixture is stirred for 30 min at room temperature. This solution is added dropwise to a solution of crude 2-chloro-3-chloromethyl-7-fluoroquinoline in DMF (30 mL) at −40° C. and the mixture is stirred for 3 hours at same temperature. After warming up to room temperature, the mixture is quenched by addition of sat. ammonium chloride solution and extracted with ethyl acetate twice. The combined organic layer is washed with water and brine, dried over magnesium sulfate, filtered and concentrated. The residue is purified by silica gel column chromatography to give 3,5-bis(trifluoromethyl)benzyl](2-chloro-7-fluoroquinolin-3-ylmethyl)(2-methyl-2H-tetrazol-5-yl)amine.

¹H-NMR (400 MHz, CDCl₃), δ (ppm): 4.19 (s, 3H), 4.90 (s, 4H), 7.34 (m, 1H), 7.65 (dd, 1H), 7.70-7.76 (m, 4H), 7.97 (s. 1H).

Step 4:

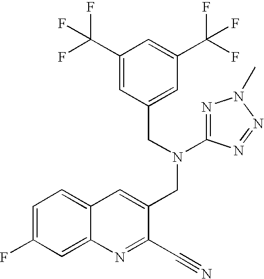

To a solution of 3,5-bis(trifluoromethyl)benzyl](2-chloro-7-fluoroquinolin-3-ylmethyl) (2-methyl-2H-tetrazol-5-yl)amine (68 mg, 0.13 mmol) in toluene (4 mL), Pottasium cyanide (26 mg, 0.39 mmol), diphenylphosphinobutane (23 mg, 0.05 mmol), palladium acetate (6 mg, 0.03 mmol) and tetramethylethylenediamine (0.2 mL, 1.3 mmol) are added at room temperature, and the mixture is stirred at 130° C. for 2.5 hours. After cooling to room temperature, the mixture is extracted with ethyl acetate. The combined organic layer is washed with brine, dried over magnesium sulfate, filtrated and concentrated. The residue is purified by silica gel column chromatography to give 3-{[(3,5-Bis-trifluoromethyl-benzyl)-(2-methyl-2H-tetrazol-5-yl)-amino]-methyl}-7-fluoroquinolin-2-carbonitrile.

¹H-NMR (400 MHz, CDCl₃), δ (ppm): 4.19 (s, 3H), 4.95 (s, 2H), 5.03 (s, 2H), 7.47 (m, 1H), 7.71 (s, 2H), 7.75-7.82 (m, 3H), 8.24 (s. 1H).

Step 5:

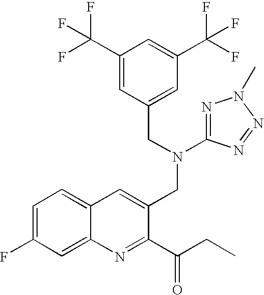

To a stirred solution of 3-{[(3,5-Bis-trifluoromethyl-benzyl)-(2-methyl-2H-tetrazol-5-yl)-amino]-methyl}-7-fluoroquinolin-2-carbonitrile (36 mg, 0.07 mmol) in THF (1 mL), 0.1 mL of EtMgBr (0.97M THF solution, 0.09 mmol) is added at 0° C. The reaction mixture is stirred at 0° C. for 30 min before adding sat. NH4Cl aq. The aqueous layer is extracted with EtOAc. The combined organic layer is washed with sat. NH4Cl aq., and brine, dried over magnesium sulfate, filtrated and concentrated in vacuo. The residue is purified by silica gel column chromatography (AcOEt/hexane=1/4) to afford 3-{[(3,5-Bis-trifluoromethyl-benzyl)-(2-methyl-2H-tetrazol-5-yl)-amino]-methyl}-2-ethylcarbonyl-7-fluoroquinoline.

¹H-NMR (400 MHz, CDCl₃), δ (ppm): 1.20 (t, 3H), 3.32 (q, 2H), 4.16 (s, 3H), 4.90 (s, 2H), 5.17 (s, 2H), 7.39 (m, 1H), 7.70-7.76 (m, 5H), 8.03 (s. 1H).

Step 6:

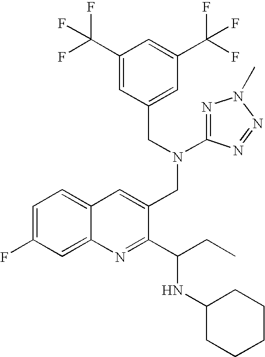

To a stirred solution of 3-{[(3,5-Bis-trifluoromethyl-benzyl)-(2-methyl-2H-tetrazol-5-yl)-amino]-methyl}-2-ethylcarbonyl-7-fluoroquinoline (26 mg, 0.05 mmol) in EtOH (1.5 mL), sodium borohydride (4 mg, 0.1 mmol) is added at 0° C. The reaction mixture was stirred at 0° C. for 30 min before adding sat. NH4Cl aq. The aqueous layer is extracted with EtOAc. The combined organic layers are washed with sat. NH4Cl aq., and brine, dried over magnesium sulfate, filtrated and concentrated in vacuo to afford the corresponding alcohol. The obtained alcohol is used without purification. To a stirred solution of the alcohol in toluene, methanesulfonyl chloride (7 μL, 0.1 mmol) and diisopropylethylamine (16 μL, 0.1 mmol) are added. The reaction mixture is stirred at room temperature for 1 h. The aqueous layer is extracted with EtOAc. The combined organic layers are washed with sat. NaHCO3 aq., and brine, dried over magnesium sulfate, filtrated and concentrated in vacuo to afford crude 1-[3-({[3,5-bis(trifluoromethyl)phenylmethyl]-(2-methyl-2H-tetrazol-5-yl)amino]methyl}-7-fluoroquinolin-2-yl)propyl]mesylate, which is used for the next reaction without purification. A suspension of 1-[3-({[3,5-bis(trifluoromethyl)phenylmethyl]-(2-methyl-2H-tetrazol-5-yl)amino]methyl}-7-fluoroquinolin-2-yl)propyl]mesylate (27 mg, 0.044 mmol), N,N-diisopropylethylamine (0.50 mL), cyclohexylamine (0.50 mL) in DMSO is stirred at 50° C. for 16 hours. The reaction mixture is cooled to room temperature, diluted with water and ethyl acetate. The organic layer is washed with brine, dried over magnesium sulfate, filtered and concentrated. The crude product is purified by silica PTLC to give 3,5-bis(trifluoromethyl)phenylmethyl]-[2-(1-cyclohexylaminopropyl)-7-fluoroquinoline-3-ylmethyl]-(2-methyl-2H-tetrazol-5-yl)amine.

ESI-MS m/z: 592 [M+1]⁺

¹H-NMR (400 MHz, CDCl₃), δ (ppm): 0.84 (t, 3H), 1.02-1.10 (m, 5H), 1.50-1.68 (m, 8H), 2.04 (m, 1H), 4.03 (m, 1H), 4.20 (s, 3H), 4.72 (d, 1H), 4.82 (s, 2H), 4.96 (d, 2H), 7.29 (m, 1H), 7.65-7.70 (m, 4H), 7.73 (s, 1H), 7.78 (s, 1H).

1. A compound of formula (I): 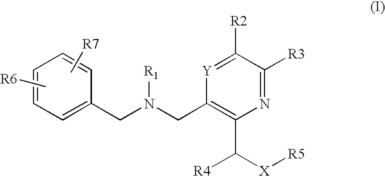
R1 is substituted or unsubstituted heterocyclyl, substituted or unsubstituted aryl, substituted or unsubstituted alkoxycarbonyl, substituted or unsubstituted alkanoyl, or substituted or unsubstituted alkyl;
R2 or R3 are independently of each other hydrogen, substituted or unsubstituted alkyl, substituted or unsubstituted alkoxy, halogen, cyano, nitro, hydroxyl, amino, NR′R″, wherein R′ and R″, independently of one another, represents hydrogen, substituted or unsubstituted alkyl, substituted or unsubstituted aryl, substituted or unsubstituted cycloalkyl, or R′ and R″ form a 5-7-membered carbocyclic ring together with the nitrogen;
or R2 and R3 may form together a 5-7-membered aromatic or heteroaromatic ring fused to the ring to which they are attached, whereby said 5-7-membered aromatic or heteroaromatic ring that may be substituted or unsubstituted;
R4 is substituted or unsubstituted alkyl, substituted or unsubstituted aryl, substituted or unsubstituted aryl alkyl or substituted or unsubstituted cycloalkyl;
X is O or NR8;
R5 or R8 are independently of each other hydrogen, substituted or unsubstituted alkyl, substituted or unsubstituted aryl, substituted or unsubstituted cycloalkyl; or
R5 and R8 can form together a 5-7-membered carbocyclic ring together with the nitrogen which ring can be substituted or unsubstituted;
R6 and R7 are independently hydrogen, alkyl, haloalkyl, halogen, cyano, nitro, hydroxy, haloalkoxy, or alkoxy; or
R6 is substituted or unsubstituted aryl or substituted or unsubstituted heteroaryl;
Y is N or CH;
or a pharmaceutically acceptable salt thereof; or an optical isomer thereof; or a mixture of optical isomers.

2. The compound according to claim 1 wherein
R1 is heterocyclyl, aryl, alkoxycarbonyl, alkanoyl, or alkyl, wherein each heterocyclyl or aryl is optionally substituted with one to three substituents selected from alkyl, haloalkyl, hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, or heterocyclyl; and wherein each alkanoyl, alkoxycarbonyl, or alkyl is optionally substituted with one to three substituents selected from hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, or heterocyclyl;
R2 or R3 are independently of each other hydrogen, alkyl, alkoxy, halogen, cyano, nitro, hydroxyl, amino, NR′R″, wherein R′ and R″, independently of one another, represents hydrogen, alkyl, aryl, cycloalkyl, or R′ and R″ form a 5-7-membered carbocyclic ring together with the nitrogen, wherein each alkyl, alkoxy, aryl or cycloalkyl may be unsubstituted or substituted with one to three substituents selected from haloalkyl, hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, or heterocyclyl,
or R2 and R3 may form together a 5-7-membered aromatic or heteroaromatic ring fused to the ring to which they are attached, whereby said 5-7-membered aromatic or heteroaromatic ring may be unsubstituted or substituted with one to three substituents selected from alkyl, haloalkyl, hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, or heterocyclyl;
R4 is alkyl, aryl, aryl alkyl or cycloalkyl, wherein each alkyl may be unsubstituted or substituted with one to three substituents selected from hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, or heterocyclyl, and wherein each aryl, aryl alkyl or cycloalkyl may be unsubstituted or substituted with one to three substituents selected from alkyl, haloalkyl, hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, or heterocyclyl;
X is O or NR8,
R5 or R8 are independently of each other hydrogen, alkyl, aryl, cycloalkyl, wherein each alkyl may be unsubstituted or substituted with one to three substituents selected from hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, heterocyclyl, or NR′R″, wherein R′ and R″, independently of one another, represents hydrogen, alkyl, aryl or cycloalkyl, or R′ and R″ form a 5-7-membered carbocyclic ring together with the nitrogen, and wherein each aryl, aryl alkyl or cycloalkyl may be unsubstituted or substituted with one to three substituents selected from alkyl, haloalkyl, hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, heterocyclyl, or NR′R″, wherein R′ and R″, independently of one another, represents hydrogen, alkyl, aryl or cycloalkyl, or R′ and R″ form a 5-7-membered carbocyclic ring together with the nitrogen, or
R5 and R8 can form together a 5-7-membered carbocyclic ring together with the nitrogen which ring can be unsubstituted or substituted with one to three substituents selected from alkyl, haloalkyl, hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, heterocyclyl, or NR′R″, wherein R′ and R″ independently of one another, represents hydrogen, alkyl, aryl or cycloalkyl, or R′ and R″ form a 5-7-membered carbocyclic ring together with the nitrogen;
R6 and R7 are independently hydrogen, alkyl, haloalkyl, halogen, cyano, nitro, hydroxy, haloalkoxy, or alkoxy; or
R6 is aryl or heteroaryl;
a pharmaceutically acceptable salt thereof; or an optical isomer thereof; or a mixture of optical isomers.

3. The compound according to claim 1, wherein
R1 is heterocyclyl, alkanoyl or alkoxycarbonyl, wherein each heterocyclyl is optionally substituted with one to three substituents selected from alkyl, hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, or heterocyclyl.

4. The compound according to claim 1, wherein
R1 is pyrimidyl, pyridyl, pyrazinyl, tetrazoyl, triazoyl, pyrazoyl, or alkoxycarbonyl, wherein each pyrimidyl, pyridyl, pyrazinyl is optionally substituted with one to three substituents selected from alkyl, hydroxy, halogen, nitro, carboxy, thiol, cyano, HSO₃—, cycloalkyl, alkenyl, alkoxy, cycloalkoxy, alkenyloxy, alkoxycarbonyl, carbamimidoyl, alkyl-S—, alkyl-SO—, alkyl-SO₂—, amino, H₂N—SO₂—, alkanoyl, or heterocyclyl.

5. The compound according to claim 1, wherein
R2 or R3 are independently of each other hydrogen, alkyl, haloalkyl, alkoxy, halogen, cyano, nitro, hydroxyl, amino, NR′R″, wherein R′ and R″, independently of one another, represent hydrogen, alkyl, aryl, cycloalkyl, or R′ and R″ form a 5-7-membered carbocyclic ring together with the nitrogen, preferably haloalkyl.

6. The compound according to claim 1, wherein one of R2 and R3 is hydrogen and the other is a moiety other than hydrogen.

7. The compound according to claim 1, wherein
R2 and R3 may form together a 5-7-membered aromatic or heteroaromatic ring fused to the ring to which they are attached, whereby said 5-7-membered aromatic or heteroaromatic ring may be unsubstituted or substituted with one to three substituents selected from alkyl, haloalkyl, hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, or heterocyclyl and wherein the aromatic or heteroaromatic ring is selected from phenyl, pyridyl, pyrimidyl, or pyrazinyl.

8. The compound according to claim 1, wherein
R4 is alkyl or cycloalkyl, wherein alkyl may be unsubstituted or substituted with one to three substituents selected from hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, or heterocyclyl;

9. The compound according to claim 1, wherein
R5 or R8 are independently of each other hydrogen, alkyl or cycloalkyl, wherein each alkyl or cycloalkyl may be unsubstituted or substituted with one to three substituents selected from hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, heterocyclyl, or NR′R″, wherein R′ and R″, independently of one another, represents hydrogen, alkyl, aryl or cycloalkyl, or R′ and R″ form a 5-7-membered carbocyclic ring together with the nitrogen; preferably R5 or R8 are independently of each other hydrogen, methyl, ethyl, cyclopentyl, or cyclohexyl.

10. The compound according to claim 1, wherein one of R5 and R8 is hydrogen and the other is a moiety other than hydrogen.

11. The compound according to claim 1, wherein
R5 and R8 can form together a 5-7-membered carbocyclic ring together with the nitrogen which ring can be unsubstituted or substituted with one to three substituents selected from alkyl, haloalkyl, hydroxy, halogen, nitro, carboxy, thiol, cyano, HSO₃—, cycloalkyl, alkenyl, alkoxy, cycloalkoxy, alkenyloxy, alkoxycarbonyl, carbamoyl, alkyl-S—, alkyl-SO—, alkyl-SO₂—, amino, H₂N—SO₂—, alkanoyl, heterocyclyl, or NR′R″, wherein R′ and R″ independently of one another, represents hydrogen, alkyl, aryl or cycloalkyl, or R′ and R″ form a 5-7-membered carbocyclic ring together with the nitrogen; and wherein the ring formed by R5 and R8 is selected from pyrrolidinyl or piperidinyl.

12. The compound according to claim 1, wherein R6 and R7 are independently hydrogen, alkyl, haloalkyl, halogen, or alkoxy.

13. The compound according to claim 1, wherein R6 and R7 are hydrogen, alkyl or haloalkyl.

14. A method of inhibiting CETP activity in a subject, comprising:
administering to the subject a therapeutically effective amount of the compound of formula (I) according to claim 1.

15. A method of treating a disorder or a disease in a subject mediated by CETP or responsive to inhibition of CETP, comprising:
administering to the subject a therapeutically effective amount of the compound of formula (I) according to claim 1.

16. The method of claim 15, wherein the disorder or the disease is selected from hyperlipidemia, arteriosclerosis, atherosclerosis, peripheral vascular disease, dyslipidemia, hyperbetalipoproteinemia, hypoalphalipoproteinemia, hypercholesterolemia, hypertriglyceridemia, familial hypercholesterolemia, cardiovascular disorder, coronary heart disease, coronary artery disease, coronary vascular disease, angina, ischemia, heart ischemia, thrombosis, cardiac infarction such as myocardial infarction, stroke, peripheral vascular disease, reperfusion injury, angioplasty restenosis, hypertension, congestive heart failure, diabetes, diabetic vascular complications, obesity or endotoxemia.

17. A pharmaceutical composition, comprising:
a therapeutically effective amount of the compound of formula (I) according to claim 1 and one or more pharmaceutically acceptable carriers.

18. A pharmaceutical composition, comprising:
a therapeutically effective amount of the compound of formula (I) according to claim 1 and
one or more therapeutically active agent selected from the group consisting of a:
(i) HMG-Co-A reductase inhibitor or a pharmaceutically acceptable salt thereof,
(ii) angiotensin II receptor antagonist or a pharmaceutically acceptable salt thereof,
(iii) angiotensin converting enzyme (ACE) Inhibitor or a pharmaceutically acceptable salt thereof,
(iv) calcium channel blocker or a pharmaceutically acceptable salt thereof,
(v) aldosterone synthase inhibitor or a pharmaceutically acceptable salt thereof,
(vi) aldosterone antagonist or a pharmaceutically acceptable salt thereof,
(vii) dual angiotensin converting enzyme/neutral endopeptidase (ACE/NEP) inhibitor or a pharmaceutically acceptable salt thereof,
(viii) endothelin antagonist or a pharmaceutically acceptable salt thereof,
(ix) renin inhibitor or a pharmaceutically acceptable salt thereof,
(x) diuretic or a pharmaceutically acceptable salt thereof, and
(xi) an ApoA-I mimic.

19-21. (canceled)
